(19) United States
(12) Patent Application Publication
(10) Pub. No.: US 2021/0322476 A1
(43) Pub. Date: Oct. 21, 2021

(54) COMPOSITIONS AND METHODS FOR IMMUNOSUPPRESSION

(71) Applicant: The Brigham and Women's Hospital, Inc., Boston, MA (US)

(72) Inventors: Anil CHANDRAKER, Westwood, MA (US); Sudipta TRIPATHI, Brookline, MA (US); Ana Maria WAAGA-GASSER, Boston, MA (US)

(21) Appl. No.: 17/312,581

(22) PCT Filed: Dec. 12, 2019
(86) PCT No.: PCT/US2019/066006

§ 371 Date: Jun. 10, 2021

Related U.S. Application Data
(60) Provisional application No. 62/778,538, filed on Dec. 12, 2018.

(51) Int. Cl. A61K 35/17; C07K 14/74; C12N 5/0783; A61P 37/06; A61K 38/20

(52) U.S. Cl. CPC A61K 35/17; C07K 14/70539; A61K 38/2013; C12N 5/0646; A61P 37/06; C12N 5/0637

(57) ABSTRACT

Provided herein is are regulatory T cells (Tregs) capable of specifically suppressing immune response against a donor alloantigen or an autoantigen, compositions thereof, and methods for producing the same. Optionally, the Tregs are used in a population including natural killer (NK) cells. Also described are related methods for using the Tregs or mixed population of Tregs and NK cells, including for promoting allograft acceptance in a transplant recipient and treating autoimmune disorders in a subject. The Tregs or mixed population of Treg and NK cells are derived from the subject's blood cells and can reduce or replace the use of broad-acting immunosuppressants.

### SEQUENCE LISTING

The instant application contains a Sequence Listing which has been submitted electronically in ASCII format and is hereby incorporated by reference in its entirety. Said ASCII copy, created on Dec. 12, 2019, is named 51329-002WO2_Sequence_Listing_12.12.19_ST25 and is 3,714 bytes in size.

### BACKGROUND OF THE INVENTION

Kidney transplantation is currently the preferred treatment for patients with end stage kidney disease (ESKD). According to the U.S. Renal Data System Annual Report, more than 660,000 Americans are being treated for ESKD. Of these patients, 468,000 are dialysis patients, and more than 193,000 have a functioning kidney transplant. Over 89,000 people with ESKD die annually, and the annual Medicare spending to treat kidney failure in the U.S. is approximately $31 billion, which is about 7% of all Medicare costs. As of 2016, there are currently over 100,000 patients awaiting a kidney transplant in the United States. The median wait time for an individual's first transplant is 3.6 years, and wait time can vary depending on health, compatibility, and availability of organs. In 2017, approximately 20,000 kidney transplants took place in the U.S., two thirds of which came from deceased donors, and one third from living donors.

Despite improvements in short term graft survival, long term graft survival has not changed significantly in recent years. Currently, organ transplantation patients receive broad spectrum immunosuppressants to prevent rejection of the donated organ. However, these immunosuppressants leave the transplant recipient vulnerable to serious infections, especially because transplant recipients are chronically maintained on the immunosuppressant drug protocol. The development of infections and cancer because of treatment with immunosuppressants is a significant problem in transplant recipients. Accordingly, there exists a need for alternative treatments to promote transplant tolerance and prevent rejection that would allow for the elimination of toxicities associated with current immunosuppressive drugs, including those that impact allograft survival.

### SUMMARY OF THE INVENTION

The invention described herein provides, inter alia, a regulatory T cell (Treg) derived from a patient specific to (i) a transplant donor alloantigen, or (ii) an autoantigen. The invention also provides methods for suppressing an immune response against an alloantigen or autoantigen, as well as methods for promoting allograft acceptance and for treating or preventing transplant rejection or an autoimmune disorder. The Tregs can also be used in a mixed population of Tregs and NK cells.

In one aspect, the invention provides an isolated regulatory T cell (Treg) including a T cell receptor (TCR) that specifically binds to (i) an alloantigen that is a human leukocyte antigen (HLA) molecule, or a fragment thereof, and is not encoded by a nucleotide sequence present in the genome of the Treg, or (ii) an autoantigen contributing to an autoimmune disorder, or a fragment thereof.

In particular embodiments, the TCR specifically binds to the HLA molecule. In some embodiments, the TCR specifically binds to a hypervariable region (HVR), e.g., a β-chain HVR of the HLA molecule. In some embodiments, the HLA molecule is an HLA-DR, HLA-DQ, HLA-DP, HLA-A, HLA-B, or HLA-C, molecule, or a fragment thereof. In particular embodiments, the HLA molecule is an HLA-DR, HLA-DQ, or HLA-DP molecule, or a fragment thereof. In certain embodiments, the HLA-DR molecule is an HLA-DR1, HLA-DR2, HLA-DR3, HLA-DR4, HLA-DR5, HLA-DR6, HLA-DR7, HLA-DR8, HLA-DR9, HLA-DR10, HLA-DR11, HLA-DR12, HLA-DR13, HLA-DR14, HLA-DR15, HLA-DR16, HLA-DR17, HLA-DR18, HLA-DR51, HLA-DR52, or HLA-DR53 molecule, or any other HLA-DR serotype as described herein or known in the art. In some embodiments, the HLA molecule, or the fragment thereof, to which the TCR specifically binds is encoded by a nucleotide sequence that is present in the genome of a donor of an organ or tissue.

In some embodiments, the Treg is capable of suppressing T effector cell (Teff) responses directed towards the alloantigen or the autoantigen. In further embodiments, the Treg is capable of suppressing Teff proliferation responses to direct allorecognition, semi-direct allorecognition, and/or indirect allorecognition.

In some embodiments, the Treg includes activating the adenosinergic signaling pathway.

In some embodiments, the Treg expresses one or more markers selected from the group consisting of CD4, CD25, CD39, CD73, FOXP3, GITR, CLTA4, ICOS, GARP, LAP, PD-1, CCR6, and CXCR3.

In another aspect, the invention features an isolated Treg including a TCR that specifically binds to (i) an alloantigen that is an HLA molecule, or a fragment thereof, and is not encoded by a nucleotide sequence present in the genome of the Treg, or (ii) an autoantigen contributing to an autoimmune disorder, or a fragment thereof; wherein the Treg has been produced by a method including (a) contacting an immune cell population comprising T cells obtained from a recipient subject with a fragment of the HLA molecule or autoantigen and an autologous antigen-presenting cell (APC); and (b) expanding the immune cell population of step (a) for a time and under conditions sufficient to form an expanded T cell line comprising a plurality of the Tregs; and, optionally (c) purifying the Tregs from the immune cell population. In some embodiments, the immune cell population of (a) further comprises natural killer (NK) cells, and, if step (c) is performed, step (c) comprises purifying the Tregs and NK cells from the immune cell population, thereby producing a mixed population of Tregs and NK cells.

In another aspect, the invention features a mixed population of cells including the Tregs of any of the preceding aspects and NK cells.

In another aspect, the invention features a composition including the Treg of any one of the preceding embodiments.

In another aspect, the invention features a composition comprising the mixed population of Tregs and NK cells of the preceding aspect.

In another aspect, the invention features a method of suppressing an immune response in a subject, the method including administering the Treg, the mixed population of Tregs and NK cells, or the pharmaceutical composition of any one of the preceding aspects to the subject. In some embodiments, the immune response is a Teff response directed towards the alloantigen or the autoantigen.

In another aspect, the invention features a method of treating or preventing transplant rejection or a method of treating an autoimmune disorder in a subject, the method including administering the Treg, the mixed population of Tregs and NK cells, or the composition of any one of the preceding aspects to the subject.

In some embodiments, the subject has an autoimmune disorder (e.g., autism, autism spectrum disorder, rheumatoid arthritis, lupus, focal segmental glomerulonephritis, or membranous nephropathy).

In some embodiments, the subject is an organ or tissue transplant recipient. In some embodiments of any of the preceding aspects, the HLA molecule, or the fragment thereof, to which the TCR specifically binds is encoded by a nucleotide sequence that is present in the genome of the donor of the organ or tissue. In further embodiments, the method further comprises reducing the dose (e.g., by 10%, by 20%, by 30%, by 40%, by 50%, by 60%, by 70%, by 80%, by 90%, or by 100%) of an immunosuppressive agent administered to the subject. Preferably, the dose of the immunosuppressive agent is reduced by up to 50% (e.g., by 10%, by 20%, by 30%, by 40%, or by 50%).

In some embodiments, the organ is a kidney, a liver, a heart, a lung, a pancreas, an intestine, a stomach, a testis, a penis, a thymus, or a face, hand, or leg vascular composite allograft. In some embodiments, the tissue includes bone, a tendon, a cornea, skin, a heart valve, nervous tissue, bone marrow, islets of Langerhans, stem cells, blood, or a blood vessel.

In another aspect, the invention features a method for producing the Treg of any one of the preceding aspects, the method including (a) contacting an immune cell population including T cells obtained from a recipient subject with a fragment of the HLA molecule or autoantigen and an autologous antigen-presenting cell (APC); and (b) expanding the immune cell population of step (a) for a time and under conditions sufficient to form an expanded T cell line including a plurality of the Tregs; and, optionally (c) purifying the Tregs from the immune cell population.

In some embodiments, the method includes repeating steps (a) and (b) more than one time. In some embodiments, the method includes repeating steps (a) and (b) more than three times, e.g., four or five times. In further embodiments, step (a) is performed about every seven to ten days.

In some embodiments, the autologous APCs are peripheral blood mononuclear cells (PBMCs), dendritic cells, macrophages, or B cells. In certain embodiments, the autologous APCs are PBMCs. In some embodiments, the PBMCs are irradiated.

In some embodiments, the immune cell population including T cells is a population of PBMCs, a population of naive T cells, or a population of purified Tregs. In particular embodiments, the immune cell population is a population of PBMCs. In further embodiments, step (a) further includes contacting the recipient subject PBMCs with IL-2. In some embodiments, the concentration of IL-2 is about 50 IU/ml to about 200 IU/ml, e.g., about 100 In other embodiments, the concentration of the fragment of the HLA molecule or autoantigen is about 25 μg/ml to about 200 μg/ml, e.g., about 50 μg/ml. In some embodiments, the fragment of the HLA molecule is a purified peptide or peptide mixture. In some embodiments, the immune cell population includes NK cells. In some embodiments, step (c) includes purifying the Tregs and NK cells from the immune cell population, thereby producing a mixed population of Tregs and NK cells.

In another aspect, the invention features a composition including: (a) the Treg of any one of the preceding aspects; and (b) a fragment of the HLA molecule or autoantigen.

In some embodiments, the composition further includes IL-2. In some embodiments, the concentration of IL-2 is about 50 IU/ml to about 200 IU/ml, e.g., about 100 IU/ml. In some embodiments, the concentration of the fragment of the HLA molecule or autoantigen is about 25 μg/ml to about 200 μg/ml, e.g., about 50 μg/ml. In some embodiments, the fragment of the HLA molecule or autoantigen is a purified peptide or peptide mixture. In some embodiments, the composition further includes NK cells.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, Ada M. Kruisbeek, David H. Margulies, Ethan M. Shevach, Warren Strobe, (eds.) John Wiley and Sons, Inc., 2003 (ISBN 0471142735, 9780471142737), the contents of which are all incorporated by reference herein in their entireties.

The term “about” as used herein when referring to a measurable value, such as an amount or concentration, is meant to encompass variations of ±10%, ±5%, ±1%, ±0.5%, or ±0.1% of the specified value as well as the specified value. For example, “about X” where X is the measurable value, is meant to include X as well as variations of ±10%, ±5%, ±1%, ±0.5%, or ±0.1% of X. A range provided herein for a measurable value may include any other range and/or individual value therein.

As used herein, the term “administration” refers to the administration of a composition (e.g., an isolated Treg, a pharmaceutical composition thereof, any additional therapeutic agent, and/or any pharmaceutical composition that includes an additional therapeutic agent) to a subject. Administration to an animal subject (e.g., to a human) may be by any appropriate route. For example, administration may be bronchial (including by bronchial instillation), buccal, enteral, parenteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, or vitreal. The administration may be systemic or local.

As used herein, “allogeneic” refers to cells, tissue, organs, nucleic acids (e.g., DNA), or polypeptides (e.g., proteins), or other molecules derived from or obtained from a different subject of the same species, e.g., a subject from the same species as a transplant recipient. An “alloantigen” refers to an antigen that occurs in some but not all members of the same species.

The term “antigen presenting cell” or “APC” refers to a cell (e.g., an immune system cell such as an accessory cell (e.g., a B cell, a dendritic cell, or a macrophage)) that displays an antigen (e.g., a foreign antigen) complexed with major histocompatibility complexes (MHCs) on its surface. In some embodiments, the APC may be a professional APC (e.g., a cell that expresses MHC class II molecules, including a B cell, a dendritic cell, or a macrophage). In other embodiments, the APC may be a non-professional APC (e.g., a cell that expresses MHC class I molecules, such as a fibroblast, a glial cell, or an endothelial cell). APCs process antigens and present them to T cells. T cells may recognize these complexes using their T cell receptors (TCRs).

As used herein, “autoantigen” or “self-antigen” is any substance normally found within a subject which, in an abnormal situation, is no longer recognized as part of the subject itself by the lymphocytes or antibodies of that subject, and is therefore attacked by the immune system as though it were a foreign substance. An autoantigen can be a naturally occurring molecule such as a protein normally produced and used by the subject itself, eliciting an immune response possibly leading to an autoimmune disease or disorder in the subject.

As used herein, an “autoimmune disease” or “autoimmune disorder” is characterized by the inability of one's immune system to distinguish between a foreign cell and a healthy cell. This results in one's immune system targeting one's healthy cells for programmed cell death.

As used herein, the term “autologous” refers to cells, tissue, organs, nucleic acids (e.g., DNA), or polypeptides (e.g., proteins) derived from or obtained from the same subject or patient.

As used herein, the term “fragment” refers to less than 100% of the amino acid sequence of a reference protein (e.g., 99%, 90%, 80%, 70%, 60%, 50%, 40%, 30%, 20%, or 10% of the reference length sequence), but including, e.g., 5, 6, 7, 8, 9, 10, 15, or more amino acids. A fragment can be of sufficient length such that a desirable function of the reference protein is maintained.

As used herein, “immune response” refers to a response made by the immune system of an organism to a substance, which includes but is not limited to foreign or self proteins. Three general types of “immune response” include mucosal, humoral, and cellular immune responses. An immune response may include at least one of the following: antibody production, inflammation, developing immunity, developing hypersensitivity to an antigen, the response of antigen-specific lymphocytes to antigen, and transplant or graft rejection.

An “immunosuppressant” or “immunosuppressive agent” is any agent that prevents, delays the occurrence of, or reduces the intensity of an immune reaction against a foreign cell in a host, particularly a transplanted cell. Examples of immunosuppressive agents include, but are not limited to, cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art.

As used herein, the term “isolated” refers to a product, compound, or composition which is separated from at least one other product, compound, or composition with which it is associated in its naturally occurring state, whether in nature or as made synthetically.

The terms “major histocompatibility complex” and “MHC” as used herein refer to a specific cluster of genes, many of which encode evolutionarily-related cell surface proteins involved in antigen presentation, which are among the most important determinants of histocompatibility. MHC molecules are also known in the art as major histocompatibility antigens. Class I MHC, or MHC-I, function mainly in antigen presentation to CD8⁺ T lymphocytes. Class II MHC, or MHC-II, function mainly in antigen presentation to CD4⁺ T lymphocytes. Class I MHC molecules are heterodimers of a heavy chain encoded in the MHC (also known as the α-chain) and β2-microglobulin (β2M). The extracellular region of the heavy chain folds into three domains (α1, α2, and α3), and β2M contributes a fourth domain. The peptide-binding site of MHC class I molecules is largely composed of the α1 and α2 domains, which form a groove that binds antigenic peptides. Class II MHC molecules are also heterodimers, but do not include β2M, and instead include an α chain and β chain, both of which are encoded in the MHC. The Class II MHC α chain is a transmembrane protein that includes extracellular α1 and α2 domains, and the β chain is a transmembrane protein that includes extracellular β1 and β2 domains. The α1 and β1 domains form the peptide-binding site of MHC class II molecules. For a review of the MHC and its functions, see, e.g. Janeway's Immunobiology, supra.

In humans, the MHC genes are referred to as “human leukocyte antigen” or “HLA” genes. For example, there are three Class I MHC α-chain genes in humans, called HLA-A, HLA-B, and HLA-C, and three pairs of Class II MHC α- and β-chain genes, called HLA-DR, HLA-DP, and HLA-DQ. The HLA-DR cluster may contain an extra β-chain gene whose product can pair with the DRα chain.

The term “organ” as used herein refers to a structure of bodily tissue that as a whole is specialized to perform a particular bodily function. Organs which are transplanted within the meaning of the invention described herein include, for example, but without limitation, a heart, a kidney, a liver, a lung, a bladder, a ureter, a stomach, an intestine (e.g., a small intestine and a large intestine), skin, a tongue, an esophagus, an endocrine gland (e.g., a pancreas, adrenal gland, salivary gland, thyroid gland, pituitary gland, and the like), bone marrow, a spleen, a thymus, a lymph node, a tendon, a ligament, a muscle, a uterus, a vagina, an ovary, a fallopian tube, a penis, a testis, a cornea, a lens, a retina, a middle ear, an outer ear, a cochlea, an iris, and a vein. Organs for transplantation can also include vascular composite allografts, such as face, hand, or leg.

The term “peripheral blood mononuclear cell,” or “PBMC,” refers to any blood cell with a round nucleus, e.g., a lymphocyte, a monocyte, or a dendritic cell.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject. A pharmaceutical composition may include an isolated Treg described herein.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and/or other problem complications commensurate with a reasonable benefit/risk ratio.

As used herein, the term “mixed population of Treg and NK cells” refers to a mixture of Tregs and NK cells that have been stimulated with the alloantigen or autoantigen and expanded according to the procedures described herein. The cells can be present in the proportion of 1:1, 1:2, 1:3, 1:4, 1:5, 1:6, 1:10, 1:20, 1:50, 1:100, 100:1, 50:1, 20:1, 10:1, 6:1, 5:1, 4:1, 3:1, or 2:1 of Treg cells to NK cells. In preferred embodiments, the cells are present in a proportion of 6:1 to 2:1 Treg cells to NK cells. As described herein, a mixed population of Treg and NK cells includes a minimal amount, e.g., less than 2% (or 1% or less, or 3%, 4%, 5%, 6%, 7%, 8%, 9%, or 10%) of the population, of other cell types.

The terms “preventing” and “prevention” refer to the administration of an agent or composition to a clinically asymptomatic individual who is susceptible to a particular adverse condition, disorder, or disease, and thus relates to the prevention of the occurrence of at least one sign or symptom of a disease. As used herein, unless indicated otherwise, the term “symptom” includes signs and symptoms.

The terms “rejection” or “transplant rejection” as used herein refers to the process or processes by which the immune response of an organ transplant recipient mounts a reaction against the transplanted organ, cell, or tissue, whether native or bioartificial, such as a recellularized tissue, sufficient to impair or destroy normal function of the organ. The immune system response can involve specific (antibody and T cell-dependent) or non-specific (phagocytic, complement-dependent, and the like) mechanisms, or both. In one example, rejection or acceptance of a kidney transplant can be measured by creatinine levels in the blood, wherein a creatinine level of ≥1.6 mg/dl indicates chronic rejection, while a creatinine level of ≤1.6 mg/dl indicates stable kidney function.

As used herein, the terms “specific binding” and “specifically binds” refer to a physical interaction between two molecules, compounds, cells, and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times, or more greater than the affinity for the third non-target entity under the same conditions. A reagent specific for a given target is one that exhibits specific binding for that target under the conditions of the assay being utilized. A non-limiting example includes an antibody, or a ligand, which recognizes and binds with a cognate binding partner (for example, a stimulatory and/or costimulatory molecule present on a T cell) protein.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, dogs, cats, non-human primates, and humans). Preferably, the subject is a human. A subject may seek or be in need of treatment, require treatment, be receiving treatment, be receiving treatment in the future, or be a human or animal who is under care by a trained professional for a particular disease or condition. The subject may be a patient (e.g., a transplant recipient).

As used herein, to “suppress” a function or activity is to reduce the function or activity when compared to otherwise same conditions except for a condition or parameter of interest, or alternatively, as compared to another condition, for example, an immune response in a subject. An “immunosuppressive” effect or response generally refers to the production or expression of cytokines or other molecules by an APC that reduces, inhibits, or prevents an immune response. When an APC results in an immunosuppressive effect on immune cells that recognize the antigen presented by the APC, the immunosuppressive effect is said to be specific to the presented antigen.

As used herein, the term “T cell” refers to a type of lymphocyte that plays a central role in cell-mediated immunity. T cells can be distinguished from other lymphocytes, such as B cells and natural killer cells, by the presence of a T cell receptor (TCR) on the cell surface. T cells do not present antigens and rely on other lymphocytes (e.g., natural killer cells, B cells, macrophages, and dendritic cells) to aid in antigen presentation. There are several subsets of T cells (e.g., T helper cells, memory T cells, regulatory T cells, cytotoxic T cells, natural killer T cells, gamma delta T cells, and mucosal associated invariant T cells), each having a distinct function.

As used herein, the terms “regulatory T cells” and “Tregs” refer to a subpopulation of immunosuppressive T cells, which are typically characterized as express the markers CD4, FOXP3, and CD25. Tregs modulate the immune system, maintain tolerance to self-antigens, prevent autoimmune disease, and also suppress the anti-tumor immune response.

By “tissue” is meant a group of cells having a similar morphology and function. Tissues capable of being transplanted within the meaning of the invention described herein include, but are not limited to, bone, a tendon, a cornea, skin, a heart valve, nervous tissue, bone marrow, islets of Langerhans, stem cells, blood, a blood vessel, cartilage, ligament, nerve, and middle ear.

The term “transplant” as used herein refers to an organ, part of an organ, tissue, engineered tissue, or a cell that has been transferred from its site of origin in one subject to a recipient site in the same or a different subject. For example, in an allograft transplant procedure, the site of origin of the transplant is in a donor individual and the recipient site is in another, recipient individual.

As used herein, a “transplant donor” is a mammal from which an organ, part of an organ, tissue, engineered tissue, or a cell is taken for transplant into a recipient. A “transplant recipient” refers to a mammal that receives an organ, part of an organ, tissue, engineered tissue, or a cell taken from a donor.

As used herein, the terms “treat,” “treatment,” “treating,” or “amelioration” refer to therapeutic treatments, wherein the object is to reverse, alleviate, ameliorate, inhibit, slow down, or stop the progression or severity of a condition associated with a disease or disorder, e.g., transplant rejection or GHVD. The term “treating” includes reducing or alleviating at least one adverse effect or symptom of a condition, disease, or disorder. Treatment is generally “effective” if one or more symptoms or clinical markers are reduced. Alternatively, treatment is effective if the progression of a disease is reduced or halted. That is, treatment includes not just the improvement of symptoms or markers, but also a cessation of, or at least slowing of, progress, or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The invention provides numerous advantages. For instance, the invention provides an immunotherapy that promotes allograft acceptance with the potential to reduce or eliminate the need for treatment with immunosuppressive drugs. The therapy can be individualized to each patient and requires no donor tissue and therefore is suitable for subjects that have received transplants from either deceased or living donors. Therapy can be initiated at any time post-transplant and is an adaptable approach for any type of solid organ transplant. Further, the invention is suitable for suppressing immune response to indirect, semi-direct, and/or direct alloantigen recognition. The invention is also useful for providing protection against both acute and chronic transplant rejection. Additionally, the invention is useful for the treatment of autoimmune disorders, e.g., by stimulating the Tregs with an autoantigen.

Another feature of the invention is that it is not broadly immunosuppressive in nature and instead promotes allograft acceptance by modulating the body's own immune response. The alloantigen- or autoantigen-specific approach is strategically safer due to lower interference with the global response to pathogens, and therefore is associated with low infectious tolerance to third party antigens.

Other features and advantages of the invention will be apparent from the following description of the preferred embodiments thereof, and from the claims.

### BRIEF DESCRIPTION OF THE DRAWINGS

FIGS. 1A-1D are a series of graphs showing the proliferation of CD4⁺ and CD4+CD25⁻ T cells from transplant recipients in response to donor-specific alloantigen. Cell proliferation was measured by the replication index, which is the average number of divisions that all cells have undergone after they had been stained by a cell proliferation dye.

FIG. 2 is a graph showing the proliferation of CD4⁺ T cells with Tregs pre- and post-stimulation with donor-specific antigen. Proliferation was measured after Tregs received one, two, or three stimulations.

FIG. 3 is a series of graphs showing the suppressive ability of Tregs regardless of the different combinations of immunosuppressive drug regimen received by the subjects.

FIG. 4 is a graph showing the difference in suppressive ability of Tregs between the specific HLA-DR allopeptide. No significant difference was observed.

FIGS. 5A-5F are a series of graphs showing the suppressive ability of the Tregs in the presence (contact dependent) and absence (contact independent) of a transwell membrane. FIG. 5A is a series of graphs showing contact dependent immunosuppression in subject 035. FIG. 5B is a series of graphs showing contact dependent immunosuppression in subject 008. FIG. 5C is a series of graphs showing contact dependent immunosuppression with Tregs and T cell clones 10E9, 1G11, 10H9, and 10B5. FIG. 5D is a series of graphs showing contact dependent immunosuppression in subject 022. FIG. 5E is a series of graphs showing contact dependent immunosuppression in subject 002. FIG. 5F is a series of graphs showing contact dependent immunosuppression in subject 036, who has two HLA mismatches (HLA-DR1 and HLA-DR15). Treg3—T-line post 3 stimulations; Treg4—T-line post 4 stimulations; Treg5—T-line post 5 stimulations.

FIG. 6 is a series of graphs showing suppressive effect of the Tregs in response to direct allorecognition and indirect allorecognition.

FIGS. 7A-7C are a series of graphs showing the suppressive effect of the Tregs in autologous and third party responders in response to donor-specific allostimulation. FIG. 7A is a series of graphs showing immune responses in subjects 038 (HLA-DR4 mismatch), 011 (HLA-DR4 mismatch), and 023 (HLA-DR15 mismatch) with Tregs from subject 038. FIG. 7B is a series of graphs showing immune responses in subjects 002 (HLA-DR1 mismatch), 035 (HLA-DR1 mismatch), and 037 (HLA-DR4 mismatch) with Tregs from subject 002. FIG. 7C is a series of graphs showing immune responses in subjects 004 (HLA-DR1 mismatch), 023 (HLA-DR15 mismatch), and 011 (HLA-DR4 mismatch) with Tregs from subject 004. It was observed that Tregs suppressed immune response specifically against donor alloantigen.

FIGS. 8A-8C are series of graphs showing the bystander suppressive effect of the Tregs. In FIG. 8A, subject 036 has HLA-DR1 and HLA-DR15 mismatches. In FIG. 8B, subject 004 has HLA-DR1 and HLA-DR15 mismatches. In FIG. 8C, subject 022 has HLA-15 and HLA-17 mismatches. Tregs were observed to demonstrate a bystander immunosuppressive effect in subjects with more than one HLA mismatch.

FIG. 9 is a series of graphs showing the effect of anti-IL-10 antibody on the immunosuppressive activity of the Tregs. It was observed that the anti-IL-10 antibody had no effect on the suppressive activity of Tregs.

FIG. 10 is a series of graphs showing the effect of A2A receptor antagonist istradefylline on the immunosuppressive activity of the Tregs. Istradefylline was shown to abrogate the suppressive activity of Tregs.

FIG. 11 are a series of graphs showing the effect of istradefylline on the immunosuppressive activity of Tregs in subjects 016, 018, 037, and 037.

FIGS. 12A-12H are a series of graphs showing the phenotypic markers associated with a characteristic Treg phenotype as analyzed by flow cytometry for Tregs generated from subjects 023, 035, 046, and 052. CD4⁺ T cells upregulated CD25 and Foxp3 while downregulating CD127. CD4⁺ T cells also upregulated GITR, CTLA4, ICOS, GARP, LAP, PD-1, CD39, CD73, CD45RA, CXCR3, and CCR6.

### DETAILED DESCRIPTION OF THE INVENTION

Disclosed herein is an individualized immunotherapy for transplant patients using a patient's regulatory T cells (Tregs) specific to a donor alloantigen(s). The Tregs are capable of suppressing T effector (Teff) immune response to the donor alloantigen, thereby promoting acceptance of the allograft without the need for immunosuppressants. This approach using Tregs generated from the patient allows for individualized therapy without requiring donor tissue. Additionally, this approach provides for the generation and use of Tregs specific against an autoantigen in treating autoimmune disorders. The use of Tregs avoids nonspecific immunosuppression, thereby protecting patients from the risk of infection resulting from immunosuppressive treatment. Furthermore, the Tregs described herein may also be used in a population comprising the Tregs and natural killer (NK cells).

Tregs are an important component of the immune system, acting as “professional” suppressors of an immune response. Their importance in the maintenance of allograft function has been shown in multiple in vitro and in vivo models (see, e.g., Duran-Struuck et al., Transplantation 101(2):274-283, 2017; Lam et al., Transplantation 101(10):2277-2287, 2017). According to the classical phenotypic description, Tregs are CD4⁺ cells that constitutively express high levels of the interleukin (IL)-2 receptor α-chain CD25 together with the transcription factor Foxp3, which is thought to be an essential component for the development and maintenance of regulatory function (see, e.g., Vaikunthanathan et al., Clin. Exp. Immunol. 189(2):197-210, 2017). Another surface marker, CD127, is inversely correlated with Foxp3 expression and can be utilized in the identification of Tregs (see, e.g., Liu et al., J. Exp. Med. 203(7):1701-11, 2006). The potential use of Tregs in therapy to induce tolerance to the allograft or as immunoregulation has led to an interest in increasing the number of Tregs including the development of different protocols for their expansion either through an antigen specific or non-specific way. In antigen-specific expansion, Tregs are typically exposed to alloantigens through a direct method of presentation of the alloantigen by donor B cells or dendritic cells, or indirect presentation through the use of self dendritic cells (see, e.g., Veerapathran et al., Blood 118(20):5671-80, 2011). Donor alloantigen-specific Tregs have been shown to be five to ten times more effective than non-specific polyclonal Tregs (see, e.g., Vaikunthanathan et al., Clin. Exp. Immunol. 189(2):197-210, 2017).

### Isolated Treg and NK Cells

The isolated Treg and NK cells of the invention are derived from the T and NK cells of a subject, e.g., a transplant recipient or a subject with an autoimmune disorder. T and NK cells useful for the invention include autologous T and NK cells (e.g., human T and NK cells) obtained from the subject to whom the cells are later to be administered after ex vivo modification and expansion, T and NK cells are typically obtained from peripheral blood that is collected from a subject by, e.g., venipuncture or withdrawal through an implanted port or catheter. Optionally, the blood can be obtained by a process including leukopheresis, in which white cells are obtained from the blood of a subject, while other blood components are returned to the subject. Blood or leukopheresis product (fresh or cryopreserved) can be processed to enrich for T cells using methods known in the art. Thus, for example, density gradient centrifugation (using, e.g., Ficoll) and/or counter-flow centrifugal elutriation can be carried out to enrich for mononuclear cells (including T cells). A T cell stimulation step employing, e.g., IL-2, can further be carried out in order to stimulate T cells and to deplete other cells. The T cells of enriched T cell preparations can then be subject to ex vivo modification.

Ire some embodiments, the Treg and NK cells described herein are specific to a donor alloantigen and are capable of suppressing immune response against a particular alloantigen. For example, the donor alloantigen can be an MHC molecule present in the transplant donor, but not in the transplant recipient. In particular, the donor alloantigen to which the Tregs and NK cells are specific can be a human leukocyte antigen (HLA) present in the transplant donor, but not in the transplant recipient. When the transplant donor and recipient have differing HLAs, this is known as an HLA mismatch. A transplant recipient may have more than one HLA mismatch with a donor. The Treg and NK cells described herein are specific to the HLA mismatch in the transplant recipient.

For example, the Treg and NK cells can be specific to an HLA-DR protein, e.g., an HLA-DR1, HLA-DR2, HLA-DR3, HLA-DR4, HLA-DR5, HLA-DR6, HLA-DR7, HLA-DR8, HLA-DR9, HLA-DR10, HLA-DR11, HLA-DR12, HLA-DR13, HLA-DR14, HLA-DR15, HLA-DR16, HLA-DR17, HLA-DR18, HLA-DR51, HLA-DR52, or HLA-DR53, protein, or any other HLA-DR serotypes known in the art. In another example, the Treg and NK cells can be specific to an HLA-DQ protein, e.g., an HLA-DQ2, HLA-DQ3, HLA-DQ4, HLA-DQ5, HLA-DQ6, HLA-DQ7, HLA-DQ8, or HLA-DQ9 peptide, or any other HLA-DQ serotypes known in the art. In another example, the Treg and NK cells can be specific to an HLA-DP protein, e.g., an HLA-DPw1, HLA-DPw2, HLA-DPw3, HLA-DPw4, HLA-DPw5, or HLA-DPw6 protein, or any other HLA-DP serotypes known in the art. In another example, the Treg and NK cells can be specific to an HLA-A peptide, e.g., an HLA-A1, HLA-A2, HLA-A3, HLA-A9, HLA-A10, HLA-A11, HLA-A19, HLA-A23, HLA-A24, HLA-A25, HLA-A26, HLA-A28, HLA-A29, HLA-A30, HLA-A31, HLA-A32, HLA-A33, HLA-A34, HLA-A36, HLA-A43, HLA-A66, HLA-A68, HLA-A69, HLA-A74, or HLA-A80 protein, or any other HLA-A serotypes known in the art. In another example, the Treg and NK cells can be specific to an HLA-B protein, e.g., an HLA-B5, HLA-B7, HLA-B8, HLA-B12, HLA-B13, HLA-B14, HLA-B15, HLA-B16, HLA-B17, HLA-B18, HLA-B21, HLA-B22, HLA-B27, HLA-B35, HLA-B37, HLA-B38, HLA-B39, HLA-B40, HLA-B41, HLA-B42, HLA-B44, HLA-B45, HLA-B46, HLA-B47, HLA-B48, HLA-B49, HLA-B50, HLA-B51, HLA-B52, HLA-B53, HLA-B54, HLA-B55, HLA-B56, HLA-B57, HLA-B58, HLA-B59, HLA-B60, HLA-B61, HLA-B62, HLA-B63, HLA-B64, HLA-B65, HLA-B67, HLA-B70, HLA-B71, HLA-B72, HLA-B73, HLA-B75, HLA-B76, HLA-B77, HLA-B78, HLA-B81, HLA-B*82, or HLA-B*83 protein, or any other HLA-B serotypes known in the art. In another example, the Treg and NK cells can be specific to an HLA-C protein, e.g., an HLA-Cw1, HLA-Cw2, HLA-Cw3, HLA-Cw4, HLA-Cw5, HLA-Cw6, HLA-Cw7, HLA-Cw8, HLA-Cw9, HLA-Cw10, or HLA-Cw11 protein, or any other HLA-C serotypes known in the art.

In another example, the Treg and NK cells described herein are specific to an autoantigen contributing to an autoimmune disorder. The autoantigen can be, for example, an autoantigen contributing to rheumatoid arthritis, lupus, or membranous nephropathy. The autoantigen can also be, for example, an autoantigen contributing autism or autism spectrum disorder.

In any of the preceding examples, the Treg and NK cells can be specific to the full-length HLA peptide or autoantigen. Alternatively, the Treg and NK cells can be specific to a fragment of the HLA peptide, e.g., the β-chain fragment of the HLA peptide, or to a fragment of the autoantigen. In particular, the Treg and NK cells can suppress a Teff immune response directed towards any of the preceding HLA peptides or autoantigens or fragments thereof.

The Tregs produced by the methods described herein can be characterized by the presence or absence of one or more additional molecular markers, which can be readily assessed by standard methods known in the art, e.g., flow cytometry. The Tregs produced by the methods described herein may express one or more of the markers selected from CD4, CD25, Foxp3, GITR, CTLA4, ICOS, GARP, LAP, PD-1, CD39, CD73, CD45RA, CXCR3 and CCR6. Additionally, the Tregs may downregulate or lack expression of CD127. For example, the Treg may have a CD4⁺CD25⁺CD127⁻ phenotype. The Treg may also have a CD4⁺CD25⁺CD39⁺ phenotype. In another example, the Treg may have a CD4⁺CD25⁺CD73⁺ phenotype. In any of the preceding examples, the Treg may also express one or more of the markers selected from GITR, CTLA4, ICOS, GARP, LAP, PD-1, CD39, CD73, CD45RA, CXCR3, and CCR6. NK cells may also be characterized by the presence or absence of one or more additional molecular markers, such as CD56 or CD16.

### Methods of Producing Treg and NK Cells

### Producing Treg and NK Cells

The Tregs of the invention are typically produced from an immune cell population (e.g., PBMCs obtained from a subject) containing T cells derived from the subject (e.g., a transplant recipient or a subject with an autoimmune disease or disorder). Optionally, the immune cell population also includes NK cells. In general, methods for stimulating T cells ex vivo are known in the art. For the methods described herein, T cells are stimulated by contacting the cells with a fragment (e.g., a β-chain fragment) of an HLA molecule, such as an HLA peptide, or an autoantigen, and an autologous APC, e.g., a PBMC, a dendritic cell, a macrophage, or a B cell. The immune cell population can be a population of PBMCs, a population of naïve T cells, or a population of isolated Tregs derived from the subject (e.g., a transplant recipient or a subject with an autoimmune disease or disorder), and optionally includes NK cells. For example, the immune cell population may be contacted with a concentration of HLA peptide or autoantigen from about 25 μg/ml to about 200 μg/ml, e.g., from about 25 μg/ml to about 150 μg/ml, from about 25 μg/ml to about 100 μg/ml, from about 25 μg/ml to about 75 μg/ml, from about 25 μg/ml to about 50 μg/ml, from about 30 μg/ml to about 200 μg/ml, from about 30 μg/ml to about 150 μg/ml, from about 30 μg/ml to about 100 μg/ml, from about 30 μg/ml to about 75 μg/ml, from about 40 μg/ml to about 200 μg/ml, from about 40 μg/ml to about 150 μg/ml, from about 40 μg/ml to about 100 μg/ml, from about 40 μg/ml to about 75 μg/ml, from about 50 μg/ml to about 200 μg/ml, from about 50 μg/ml to about 150 μg/ml, from about 50 μg/ml to about 100 μg/ml, or from about 50 μg/ml to about 75 μg/ml. In one particular example, the concentration of HLA peptide or autoantigen is 50 μg/ml.

To expand the T cell line, the immune cell population may be stimulated in the presence of IL-2. The concentration of IL-2 used for this method can be, for example, from about 50 IU/ml to about 200 IU/ml, e.g., from about 50 IU/ml to about 150 IU/ml, from about 50 IU/ml to about 100 IU/ml, from about 70 IU/ml to about 200 IU/ml, from about 70 IU/ml to about 150 IU/ml, from about 100 IU/ml to about 200 IU/ml, from about 100 IU/ml to about 150 IU/ml, or from about 150 IU/ml to about 200 IU/ml. In one particular example, the concentration of IL-2 is 100 IU/ml.

The immune cell population can be stimulated with the HLA peptide or autoantigen and autologous APC in the presence of IL-2 once. In other instances, the cells are stimulated more than once, e.g., two, three, four, or five times. The time interval between each stimulation is, e.g., between seven to ten days, e.g., seven, eight, nine, or ten days.

The methods described herein for providing Tregs can be performed on a population of T cells, or an immune cell population including both Tregs and NK cells. In some embodiments, the Tregs are subsequently purified from the population of T cells, or from the immune cell population. In further embodiments, a mixed population of Tregs and NK cells is purified from the immune cell population. Methods for isolating Tregs and NK cells are known in the art. For example, Tregs can be purified from the mixed population using many commercially available isolation kits (lab scale isolation) as well as a FACS cell sorter (GMP isolation). In standard preparations described herein, Tregs are purified and such purification typically includes NK cells.

### HLA Molecules

As described above, the Tregs useful for treating or preventing transplant rejection or promoting allograft acceptance are specific to an alloantigen present in an organ or tissue transplant donor but not in the recipient, e.g., an HLA protein. An HLA protein found in the donor but not in the recipient is referred to as an HLA protein mismatch. These Tregs recognizing a mismatched HLA protein can be produced by contacting the Tregs with one or more HLA peptide fragments, which may be overlapping or non-overlapping. Such HLA peptide fragments are generated from the portion of the mismatched HLA protein sequence that is present in the donor HLA protein, but not in the recipient's. For example, HLA peptides can be synthesized based on the sequence of, e.g., the hypervariable region of a 8-chain sequence of any known HLA serotype (e.g., any HLA serotype described above), or a fragment thereof. In some embodiments, the HLA peptide fragment is generated from the HLA-DR8 sequence of UniProt Accession Nos.: P04229, P01912, P13760, P13761, Q30134, Q9TQE0, Q30167, P20039, Q951E3, Q5Y7A7, Q9GIY3, P01911, or Q29974. The HLA fragment can be a peptide about 10-100, 15-50, or 18-22 amino acids long. A table of known HLA genotypes and their corresponding serotypes is provided in Table 1.

TABLE 1
HLA class I and class II genotypes and serotypes

| Genotype | HLA Serotype |
|---|---|
| A*01 | A1 |
| A*0101 | A1 |
| A*0102 | A1 |
| A*0201 | A2 |
| A*01011, A*02012 | |
| A*0202 | A2 |
| A*0203 | A203 |
| A*0204 | A2 |
| A*0205 | A2 |
| A*0206 | A2 |
| A*0207 | A2 |
| A*0208 | A2 |
| A*0209 | A2 |
| A*0210 | A210 |
| A*0211 | A2 |
| A*0212 | A2 |
| A*0213 | A2 |
| A*0214 | A2 |
| A*02142 | |
| A*0216 | A2 |
| A*0217 | A2 |
| A*01171, A*02172 | |
| A*0218 | A2 |
| A*0220 | A2 |
| A*0221 | A2 |
| A*0222 | A2 |
| A*0224 | A2 |
| A*0225 | A2 |
| A*0229 | A2 |
| A*03 | A3 |
| A*0301 | A3 |
| A*03011, A*03012, | |
| A*03013 | |
| A*0302 | A3 |
| A*0304 | A3 |
| A*1101 | A11 |
| A*1102 | A11 |
| A*1103 | A11 |
| A*1104 | A11 |
| A*1105 | A11 |
| A*2301 | A23(9) |
| A*24 | A24(9) |
| A*2402 | A24(9) |
| A*2402101, A*24021102L, | |
| A*24022 | |
| A*2403 | A2403 |
| A*2404 | A24(9) |
| A*2405 | A24(9) |
| A*2406 | A24(9) |
| A*2407 | A24(9) |
| A*2408 | A24(9) |
| A*2413 | A24(9) |
| A*2414 | A24(9) |
| A*2501 | A25(10) |
| A*2601 | A26(10) |
| A*2602 | A26(10) |
| A*2603 | A26(10) |
| A*2604 | A10 |
| A*2605 | A26(10) |
| A*2606 | A26(10) |
| A*2607 | A26(10) |
| A*2608 | A26(10) |
| A*2610 | A10 |
| A*2901 | A29(19) |
| A*2902 | A29(19) |
| A*3001 | A30(19) |
| A*3002 | A30(19) |
| A*3003 | A30(19) |
| A*3004 | A30(19) |
| A*3101 | A31(19) |
| A*31012 | |
| A*3201 | A32(19) |
| A*3202 | A32(19) |
| A*3301 | A33(19) |
| A*3303 | A33(19) |
| A*3401 | A34(10) |
| A*3402 | A34(10) |
| A*3601 | A36 |
| A*4301 | A43 |
| A*6601 | A66(10) |
| A*6602 | A66(10) |
| A*6801 | A68(28) |
| A*68011, A*68012 | |
| A*6802 | A68(28) |
| A*6803 | A28 |
| A*68031, A*68032 | |
| A*6808 | A68(28) |
| A*6901 | A69(28) |
| A*7401 | A74(19) |
| A*7402 | A74(19) |
| A*7403 | A19 |
| A*8001 | A80 |
| B*07 | B*07 |
| B*0702 | B7 |
| B*07021, B*07022, | |
| B*07023 | |
| B*0703 | B703 |
| B*0704 | B7 |
| B*0705 | B7 |
| B*0706 | B7 |
| B*0707 | B7 |
| B*0709 | B7 |
| B*0711 | B7 |
| B*08 | B*08 |
| B*0801 | B8 |
| B*0802 | B8 |
| B*0803 | B8 |
| B*0806 | B8 |
| B*1301 | B13 |
| B*1302 | B13 |
| B*14 | B*14 |
| B*1401 | B64(14) |
| B*1402 | B65(14) |
| B*1501 | B62(15) |
| B*1501101, B*15012 | |
| B*1502 | B75(15) |
| B*1503 | B72(70) |
| B*1504 | B62(15) |
| B*1505 | B62(15) |
| B*1506 | B62(15) |
| B*1507 | B62(15) |
| B*1508 | B75(15) |
| B*1509 | B70 |
| B*1510 | B71(70) |
| B*1511 | B75(15) |
| B*1512 | B76(15) |
| B*1513 | B77(15) |
| B*1514 | B76(15) |
| B*1515 | B62(15) |
| B*1516 | B63(15) |
| B*1517 | B63(15) |
| B*1518 | B71(70) |
| B*1519 | B76(15) |
| B*1521 | B75(15) |
| B*1522 | B35 |
| B*1524 | B62(15) |
| B*1525 | B62(15) |
| B*1527 | B62(15) |
| B*1528 | B15 |
| B*1529 | B15 |
| B*1530 | B75(15) |
| B*1531 | B75(15) |
| B*1532 | B62(15) |
| B*1533 | B15 |
| B*1534 | B15 |
| B*1535 | B15 |
| B*1545 | B62(15) |
| B*1546 | B72(70) |
| B*1548 | B62(15) |
| B*18 | B*18 |
| B*1801 | B18 |
| B*1802 | B18 |
| B*1803 | B18 |
| B*1805 | B18 |
| B*1806 | B18 |
| B*27 | B*27 |
| B*2701 | B27 |
| B*2702 | B27 |
| B*2703 | B27 |
| B*2704 | B27 |
| B*2705 | B27 |
| B*27052, B*27053 | |
| B*2706 | B27 |
| B*2707 | B27 |
| B*2708 | |
| B*2709 | B27 |
| B*2710 | B27 |
| B*2711 | B27 |
| B*2712 | B27 |
| B*2713 | B27 |
| B*35 | B*35 |
| B*3501 | B35 |
| B*3502 | B35 |
| B*3503 | B35 |
| B*3504 | B35 |
| B*3505 | B35 |
| B*3506 | B35 |
| B*3507 | B35 |
| B*3508 | B35 |
| B*3509 | B35 |
| B*35091, B*35092 | |
| B*3511 | B35 |
| B*3512 | B35 |
| B*3513 | B35 |
| B*3514 | B35 |
| B*3515 | B35 |
| B*3517 | B35 |
| B*3518 | B35 |
| B*3519 | B35 |
| B*3520 | B35 |
| B*3526 | B35 |
| B*3527 | B35 |
| B*3701 | B*37 |
| B*3702 | B*37 |
| B*3801 | B38(16) |
| B*3802 | B38(16) |
| B*38021, B*38022 | |
| B*3803 | B16 |
| B*3901 | B3901 |
| B*39011, B*39013 | |
| B*3902 | B3902 |
| B*39031, B*39022 | |
| B*3903 | B39(16) |
| B*3904 | B39(16) |
| B*3905 | B39(16) |
| B*3906 | B39(16) |
| B*39061, B*39062 | |
| B*3908 | B39(16) |
| B*3909 | B39(16) |
| B*3910 | B39(16) |
| B*3912 | B39(16) |
| B*3913 | B39(16) |
| B*40 | B40 |
| B*4001 | B60(40) |
| B*4002 | B61(40) |
| B*4003 | B60(40) |
| B*4004 | B40 |
| B*4005 | B4005 |
| B*4006 | B61(40) |
| B*4009 | B61(40) |
| B*4010 | B60(40) |
| B*4011 | B40 |
| B*4012 | B40 |
| B*4016 | B61(40) |
| B*4101 | B41 |
| B*4102 | B41 |
| B*4201 | B42 |
| B*4202 | B42 |
| B*44 | B44(12) |
| B*4402 | B44(12) |
| B*4403 | B44(12) |
| B*44031, B*44032 | |
| B*4404 | B44(12) |
| B*4405 | B44(12) |
| B*4406 | B44(12) |
| B*4407 | B44(12) |
| B*4408 | B44(12) |
| B*4409 | B12 |
| B*4501 | B45(12) |
| B*4601 | B46 |
| B*4701 | B47 |
| B*4702 | B47 |
| B*4801 | B48 |
| B*4802 | B48 |
| B*4805 | B48 |
| B*4901 | B49(21) |
| B*5001 | B50(21) |
| B*51 | |
| B*5101 | B51(5) |
| B*51011, B*51012 | |
| B*5102 | B5102 |
| B*51021, B*51022 | |
| B*5103 | B5103 |
| B*5104 | B53like |
| B*5105 | B51(5) |
| B*5106 | B5 |
| B*5107 | B51(5) |
| B*5108 | B51(5) |
| B*5109 | B51(5) |
| B*5116 | B52(5) |
| B*52 | |
| B*5201 | B52(5) |
| B*52011, B*52012 | |
| B*5301 | B53 |
| B*5401 | B54(22) |
| B*5501 | B55(22) |
| B*5502 | B55(22) |
| B*5503 | B67 |
| B*5504 | B55(22) |
| B*5505 | B22 |
| B*5507 | B54(22) |
| B*5601 | B56(22) |
| B*5602 | B56(22) |
| B*5604 | B56(22) |
| B*5701 | B57(17) |
| B*5702 | B57(17) |
| B*5703 | B57(17) |
| B*5704 | B57(17) |
| B*58 | |
| B*5801 | B58(17) |
| B*5802 | B58(17) |
| B*5901 | B59 |
| B*6701 | B67 |
| B*67011, B*67012 | |
| B*7301 | B73 |
| B*7801 | B78 |
| B*7802 | B78 |
| B*78021, B*78022 | |
| B*8101 | B81 |
| B*8201 | B822x45 |
| Cw*0102 | Cw1 |
| Cw*0103 | Cw1 |
| Cw*0104 | |
| Cw*0202 | Cw2 |
| Cw*02021, Cw*02022, | |
| Cw*02023, Cw*02024 | |
| Cw*0203 | |
| Cw*0204 | |
| Cw*0301 | Name |
| | abandoned |
| Cw*0302 | Cw10(w3) |
| Cw*0303 | Cw9(w3) |
| Cw*03031, Cw*03032 | |
| Cw*0304 | Cw10(w3) |
| Cw*03041, Cw*03042 | Cw10(w3) |
| Cw*0305 | |
| Cw*0306 | |
| Cw*0307 | Cw3 |
| Cw*0308 | |
| Cw*0309 | Cw3 |
| Cw*0310 | Cw3 |
| Cw*0311 | |
| Cw*0312 | |
| Cw*0401 | Cw4 |
| Cw*04011, Cw*04012 | Cw4 |
| Cw*0402 | Cw4 |
| Cw*0403 | |
| Cw*0404 | |
| Cw*0405 | |
| Cw*0406 | |
| Cw*0407 | |
| Cw*0408 | |
| Cw*0501 | Cw5 |
| Cw*0502 | Cw5 |
| Cw*0503 | |
| Cw*0504 | |
| Cw*0601 | Name |
| | abandoned |
| Cw*0602 | Cw6 |
| Cw*0603 | |
| Cw*0604 | |
| Cw*0605 | Cw6 |
| Cw*0606 | |
| Cw*0607 | |
| Cw*0701 | Cw7 |
| Cw*0702 | Cw7 |
| Cw*0703 | |
| Cw*0704 | Cw7 |
| Cw*0705 | |
| Cw*0706 | Cw7 |
| Cw*0707 | |
| Cw*0708 | |
| Cw*0709 | |
| Cw*0710 | |
| Cw*0711 | |
| Cw*0712 | Cw7 |
| Cw*0713 | |
| Cw*0714 | Cw7 |
| Cw*0801 | Cw8 |
| Cw*0802 | Cw8 |
| Cw*0803 | Cw8 |
| Cw*0804 | Cw8 |
| Cw*0805 | |
| Cw*0806 | |
| Cw*0807 | |
| Cw*0808 | |
| Cw*0809 | |
| Cw*1202 | |
| Cw*1203 | |
| Cw*1204 | |
| Cw*1205 | |
| Cw*1206 | |
| Cw*1207 | |
| Cw*1301 | |
| Cw*1402 | |
| Cw*1403 | |
| Cw*1404 | |
| Cw*1502 | |
| Cw*1503 | |
| Cw*1504 | |
| Cw*1505 | |
| Cw*1506 | |
| Cw*1507 | |
| Cw*1508 | |
| Cw*1509 | |
| Cw*1510 | |
| Cw*1601 | |
| Cw*1602 | |
| Cw*1604 | |
| Cw*1701 | |
| Cw*1702 | |
| Cw*1703 | |
| Cw*1801 | |
| Cw*1802 | |
| DPA1*0102/DPB1*0201 | DPw2 |
| DPA1*0103/DPB1*0201 | DPw2 |
| DPA1*0201/DPB1*0401 | DPw4 |
| DPA1*0201/DPB1*0901 | |
| DPB1*0101 | DPw1 |
| DPB1*01011, DPB1*01012 | |
| DPB1*0201 | DPw2 |
| DPB1*02012, DPB1*01013 | |
| DPB1*0301 | DPw3 |
| DPB1*0401 | DPw4 |
| DPB1*0402 | DPw4 |
| DPB1*0501 | DPw5 |
| DPB1*0601 | DPw6 |
| DQA1*0101/DQB1*0501 | DQ5(1) |
| DQA1*0102/DQB1*0602 | DQ6(1) |
| DQA1*0301/DQB1*0301 | DQ7(3) |
| DQA1*0301/DQB1*0302 | DQ8(3) |
| DQA1*0501/DQB1*0201 | DQ2 |
| DQA1*0501/DQB1*0301 | DQ7(3) |
| DQB1*0201 | DQ2 |
| DQB1*0203 | DQ2 |
| DQB1*0301 | DQ7(3) |
| DQB1*03011, DQB1*03012 | |
| DQB1*0302 | DQ8(3) |
| DQB1*03032 | DQ9(3) |
| DQB1*0304 | DQ7(3) |
| DQB1*0306 | DQ3 |
| DQB1*0401 | DQ4 |
| DQB1*0402 | DQ4 |
| DQB1*0501 | DQ5(1) |
| DQB1*0502 | DQ5(1) |
| DQB1*0503 | DQ5(1) |
| DQB1*05031, DQB1*05032 | |
| DQB1*0601 | DQ6(1) |
| DQB1*06011, | |
| DQB1*06012, DQB1*06013 | |
| DQB1*0602 | DQ6(1) |
| DQB1*0603 | DQ6(1) |
| DQB1*0604 | DQ6(1) |
| DQB1*0605 | DQ6(1) |
| DQB1*06051, DQB1*06052 | |
| DQB1*0609 | DQ6(1) |
| DQB1*0611 | DQ1 |
| DQB1*06111, DQB1*06112 | |
| DQB1*0612 | DQ1 |
| DQB1*0614 | DQ6(1) |
| DQB2*0202 | DQ2 |
| DRB1*0101 | DR1 |
| DRB1*0102 | DR1 |
| DRB1*01021, DRB1*01022 | |
| DRB1*0103 | DR103 |
| DRB1*0104 | DR1 |
| DRB1*0301 | DR17(3) |
| DRB1*03011, DRB1*03012 | |
| DRB1*0301 or DRB3*0201 | DR17(3) |
| DRB1*0302 | DR18(3) |
| DRB1*03021, DRB1*03022 | |
| DRB1*0303 | DR18(3) |
| DRB1*0304 | DR17(3) |
| DRB1*0305 | DR3 |
| DRB1*0306 | DR3 |
| DRB1*0401 | DR4 |
| DRB1*04011, DRB1*04012 | |
| DRB1*0401 or DRB4 | DR4 |
| DRB1*0401 or DRB4*0101 | DR4 |
| DRB1*0402 | DR4 |
| DRB1*0402 or DRB4 | DR4 |
| DRB1*0404 | DR4 |
| DRB1*04041, DRB1*04042 | |
| DRB1*0404 or DRB4 | DR4 |
| DRB1*0405 | DR4 |
| DRB1*04051, DRB1*04052 | |
| DRB1*0405 or DRB4 | DR4 |
| DRB1*0405 or DRB4*0101 | DR4 |
| DRB1*0406 | DR4 |
| DRB1*0407 | DR4 |
| DRB1*0407 or DRB4 | DR4 |
| DRB1*0408 | DR4 |
| DRB1*0409 | DR4 |
| DRB1*0410 | DR4 |
| DRB1*0411 | DR4 |
| DRB1*0413 | DR4 |
| DRB1*0414 | DR4 |
| DRB1*0415 | DR4 |
| DRB1*0416 | DR4 |
| DRB1*0417 | DR4 |
| DRB1*0419 | DR4 |
| DRB1*0420 | DR4 |
| DRB1*0421 | DR4 |
| DRB1*0422 | DR4 |
| DRB1*0423 | DR4 |
| DRB1*0424 | DR4 |
| DRB1*0425 | DR4 |
| DRB1*0426 | DR4 |
| DRB1*0428 | DR4 |
| DRB1*0429 | DR4 |
| DRB1*0701 | DR7 |
| DRB1*0703 | DR7 |
| DRB1*0801 | DR8 |
| DRB1*0802 | DR8 |
| DR*08021, DR*08022 | |
| DRB1*08032 | DR8 |
| DRB1*0804 | DR8 |
| DRB1*08041, | |
| DRB1*08042, DRB1*08043 | |
| DRB1*0805 | DR8 |
| DRB1*0806 | DR8 |
| DRB1*0807 | DR8 |
| DRB1*0809 | DR8 |
| DRB1*0810 | DR8 |
| DRB1*0811 | DR8 |
| DRB1*0812 | DR8 |
| DRB1*0814 | DR8 |
| DRB1*0816. | DR8 |
| DRB1*0817 | DR8 |
| DRB1*0901 or DRB4*0101 | DR9 |
| DRB1*1001 | DR10 |
| DRB1*1101 | DR11(5) |
| DRB1*11011, | |
| DRB1*11012, DRB1*11013 | |
| DRB1*1101 or DRB3*0202 | DR11(5) |
| DRB1*1102 | DR11(5) |
| DRB1*1103 | DR11(5) |
| DRB1*1104 | DR11(5) |
| DRB1*11041, DRB1*11042 | |
| DRB1*1105 | DR11(5) |
| DRB1*1106 | DR11(5) |
| DRB1*1107 | DR11(5) |
| DRB1*1108 | DR11(5) |
| DRB1*11081, DRB1*11082 | |
| DRB1*1109 | DR11(5) |
| DRB1*1111 | DR11(5) |
| DRB1*1113 | DR11(5) |
| DRB1*1114 | DR11(5) |
| DRB1*1116 | DR11(5) |
| DRB1*1120 | DR11(5) |
| DRB1*1121 | DR11(5) |
| DRB1*1123 | DR11(5) |
| DRB1*1125 | DR11(5) |
| DRB1*1126 | DR11(5) |
| DRB1*1127 | DR11(5) |
| DRB1*1129 | DR11(5) |
| DRB1*1201 or DRB3 | DR12(5) |
| DRB1*1202 | DR12(5) |
| DRB1*12021, DRB1*12022 | |
| DRB1*1203 | DR12(5) |
| DRB1*12032 | |
| DRB1*1205 | DR12(5) |
| DRB1*1206 | DR12(5) |
| DRB1*1301 | DR13(6) |
| DRB1*1301 or DRB3*0101 | DR13(6) |
| DRB1*1302 | DR13(6) |
| DRB1*1302 or DRB3*1301 | DR13(6) |
| DRB1*1303 | DR13(6) |
| DRB1*13031, DRB1*13032 | |
| DRB1*1304 | DR13(6) |
| DRB1*1305 | DR13(6) |
| DRB1*1306 | DR13(6) |
| DRB1*1307 | DR13(6) |
| DRB1*13071, DRB1*13072 | |
| DRB1*1308 | DR13(6) |
| DRB1*1310 | DR13(6) |
| DRB1*1311 | DR13(6) |
| DRB1*1312 | DR13(6) |
| DRB1*1314 | DR13(6) |
| DRB1*1316 | DR13(6) |
| DRB1*1317 | DR13(6) |
| DRB1*1318 | DR13(6) |
| DRB1*1320 | DR13(6) |
| DRB1*1327 | DR13(6) |
| DRB1*1329 | DR6 |
| DRB1*1401 | DR14(6) |
| DRB1*1402 | DR14(6) |
| DRB1*1403 | DR1403 |
| DRB1*1404 | DR1404 |
| DRB1*1405 | DR14(6) |
| DRB1*1407 | DR14(6) |
| DRB1*1411 | DR14(6) |
| DRB1*1412 | DR14(6) |
| DRB1*1413 | DR14(6) |
| DRB1*1414 | DR14(6) |
| DRB1*1417 | DR6 |
| DRB1*1418 | DR6 |
| DRB1*1419 | DR14(6) |
| DRB1*1420 | DR14(6) |
| DRB1*1421 | DR13(6) |
| DRB1*1426 | DR14(6) |
| DRB1*1427 | DR14(6) |
| DRB1*1429 | DR14(6) |
| DRB1*1501 | DR15(2) |
| DRB1*15011, DRB1*15012 | |
| DRB1*1501 or DRB5*0101 | DR15(2) |
| DRB1*1502 | DR15(2) |
| DRB1*15021, | |
| DRB1*15022, DRB1*15023 | |
| DRB1*1503 | DR15(2) |
| DRB1*1504 | DR2 |
| DRB1*1505 | DR15(2) |
| DRB1*1506 | DR15(2) |
| DRB1*1508 | DR2 |
| DRB1*1601 | DR16(2) |
| DRB1*16011, DRB1*16012 | |
| DRB1*1602 | DR16(2) |
| DRB116021, DRB116022 | |
| DRB1*1603 | DR2 |
| DRB1*1604 | DR16(2) |
| DRB1*1605 | DR2 |
| DRB3*0101 | DR52 |
| DRB3*01011, | |
| DRB3*01012, | |
| DRB3*01013, DRB3*0104 | |
| DRB3*0201 | DR52 |
| DRB3*0202 | DR52 |
| DRB3*0203 | DR52 |
| DRB3*0207 | DR52 |
| DRB3*0208 | DR52 |
| DRB3*0301 | DR52 |
| DRB3*0302 | DR52 |
| DRB4*01 | DR53 |
| DRB4*01011 | DR53 |
| DRB4*0103 | DR53 |
| DRB4*0103101, | |
| DRB4*01032 | |
| DRB4*0105 | DR53 |
| DRB5*0101 | DR51 |
| DRB5*01011, DRB5*01012 | |
| DRB5*0102 | DR51 |
| DRB5*0107 | DR51 |
| DRB5*0202 | DR51 |

Any other HLA proteins, and peptide fragments thereof, in addition to those listed above, can also be useful for preparing the Tregs of the invention described herein. Peptides can be readily synthesized by methods known to one of skill in the art (e.g., solid phase synthesis), or they can be synthesized by or obtained from a variety of commercial sources. One or more HLA peptide fragments corresponding to an HLA protein can be used to stimulate the Tregs as described herein. Exemplary HLA-DR peptide fragment sequences can be found in Vella et al., Transplantation. 27; 64(6):795-800, 1997, which is incorporated herein by reference in its entirety.

In one working example, at least one HLA-DR protein mismatch is identified, wherein the HLA-DR protein is found in a transplant donor, but not in a recipient. A panel of HLA-DR peptide fragments based on the mismatched HLA-DR protein(s) is synthesized where the peptides are unique to the mismatched HLA-DR protein of the donor and do not overlap with the HLA-DR protein of the recipient. The peptide fragments are synthesized based on the 8-chain hypervariable region of the mismatched HLA-DR protein and can, for example, correspond to the following sequences:

TABLE 2
Exemplary β-chain HLA-DR peptide fragment
sequences
HLA-DRB1*0101

| | | |
|---|---|---|
| Peptide 1 | RFLWQLKFECHFFNGTTERVR | SEQ ID NO: 1 |
| Peptide 2 | TERVRLLERCIYNQEESVRFD | SEQ ID NO: 2 |
| Peptide 3 | SDVGEYRAVTELGRPDAEYWN | SEQ ID NO: 3 |
| Peptide 4 | SQKDLLEQRRAAVDTYCR | SEQ ID NO: 4 |
| Peptide 5 | HNYGVGESFTVQRR | SEQ ID NO: 5 |

HLA-DRB1*1501

| | | |
|---|---|---|
| Peptide 1 | GDTRPRFLWQPKRECHFFNG | SEQ ID NO: 6 |
| Peptide 2 | ERVRFLDRYFYNQEESVRF | SEQ ID NO: 7 |
| Peptide 3 | DSDVGEFRAVTELGRPDAEY | SEQ ID NO: 8 |
| Peptide 4 | WNSQKDILEQARAAVDTYCR | SEQ ID NO: 9 |
| Peptide 5 | HNYGVVESFTVQRR | SEQ ID NO: 10 |

HLA-DRB1*0301

| | | |
|---|---|---|
| Peptide 1 | RFLEYSTSECHFFNGT | SEQ ID NO: 11 |
| Peptide 2 | ERVRYLDRYFHNQEENVRFD | SEQ ID NO: 12 |
| Peptide 3 | DSDVGEFRAVTELGRPDAEY | SEQ ID NO: 13 |
| Peptide 4 | SQKDLLEQKRGRVDNYCR | SEQ ID NO: 14 |
| Peptide 5 | HNYGVVESFTVQRR | SEQ ID NO: 15 |

While the sequences in Table 2 are provided as examples of peptide fragments that may be used in the invention, other peptide fragments may be synthesized according to the procedures described herein. The peptide fragments provided in Table 2 is not intended to be construed as limiting to the scope of HLA peptide fragment sequences useful for the procedures for generating the Tregs as described herein.

### Exemplary Mechanisms of Suppression

Several contact independent and dependent mechanisms have been described that contribute to the function of Tregs, often working simultaneously. Without being bound by theory, the Tregs described herein may suppress an immune response via one or more of the mechanisms described below.

Contact independent mechanisms that have been described include anti-inflammatory cytokine production (e.g., IL-10, IL-35, and TGF-β) (see, e.g., Maloy et al., J. Exp. Med. 197(1):111-9, 2003) and transfer of miRNA that can silence specific genes in T cells via exosomes, preventing proliferation as well as cytokine production (see, e.g., Okoye et al., Immunity 41(1):89-103, 2014).

Contact dependent mechanisms include, without limitation, the interaction of CTLA-4 with its ligands B7.1 and B7.2 on APCs, leading to a negative signal preventing T cell activation (see, e.g., Vasu et al., J. Immunol. 173(4):2866-76, 2004 and DiPaolo et al., J. Immunol. 179(7):4685-93, 2007); cell surface LAG-3 expression, which binds to MHC class II molecules and prevents the maturation and the ability of APCs to activate effector T cells; the expression of membrane-bound active TGFβ-1 on the Treg population (see, e.g., Savage et al., J. Immunol. 181(3):2220-6, 2008); the induction of apoptosis via engagement of CTLA-4 and programed cell death 1 (PD-1) (see, e.g., Francisco et al., J. Exp. Med. 206(13):3015-29, 2009); granzyme A/B expression (see, e.g., Grossman et al., Blood 104(9):2840-8, 2004); IL-2 deprivation (see, e.g., Pandiyan et al., Nat. Immunol. 8(12):1353-62, 2007); and through the disruption of the adenosinergic metabolic pathway.

Suppression by human Tregs via the adenosinergic pathway involves sequentially converting ATP into AMP and adenosine, which binds to A2a receptors on effector T cells. This activates an immunoinhibitory loop through the elevation of cytoplasmic cAMP, which causes a reduction in the production of pro-inflammatory cytokines and proliferation (see, e.g., Mandapathil et al., J. Biol. Chem. 285(10):7176-86, 2010).

### Pharmaceutical Compositions

The Tregs described herein may be incorporated into a vehicle for administration into a subject, such as a human patient receiving an organ, tissue, or cell transplant, or a patient with an autoimmune disorder. Pharmaceutical compositions containing Treg cells can be prepared using methods known in the art. Furthermore, the pharmaceutical composition can include a mixed population of Tregs and NK cells. Such compositions can be prepared using a wide variety of pharmaceutically acceptable carriers, as determined to be appropriate by those of skill in the art (see, for example, Gennaro, Remington: The Science and Practice of Pharmacology 22nd edition, Allen, L. Ed. (2013); Ansel et al., Pharmaceutical Dosage Forms and Drug Delivery Systems, 7^{th} ed., Lippincott, Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

### Therapeutic Methods and Uses

The Tregs described herein, optionally in a population including NK cells, are useful for suppressing an immune response to promote allograft acceptance in patients receiving organ or tissue transplants, or for treating or preventing transplant rejection. The organ may be any organ that can be transplanted including, but not limited to, a heart, a kidney, a liver, a lung, a bladder, a ureter, a stomach, an intestine (e.g., a small intestine or a large intestine), skin, a tongue, an esophagus, an endocrine gland (e.g., pancreas, adrenal gland, salivary gland, thyroid gland, pituitary gland, and the like), bone marrow, a spleen, a thymus, a lymph node, a tendon, a ligament, a muscle, a uterus, a vagina, an ovary, a fallopian tube, a testis, a penis, a cornea, a lens, a retina, a middle ear, an outer ear, a cochlea, an iris, and a vein. Organs that may be transplanted also include vascular composite allografts, e.g., face, hand, or leg. The tissue may be any tissue that can be transplanted including, but not limited to, a bone, bone marrow, islets of Langerhans, stem cells, blood, blood vessels, nervous tissue, cartilage, tendon, ligament, cornea, heart valve, nerve and/or vein, middle ear, cultured tissue (for example, differentiated cells that may function as an organ or a tissue), and/or 3D engineered tissues. The cell may be any cell that can be transplanted (for example, stem cells (e.g., hematopoietic stem cells)).

There is currently a variety of accepted protocols for promoting tolerance of a donor organ or tissue in a transplant recipient. Generally, these protocols include the administration of immunosuppressive agents in order to prevent recipient rejection of the transplanted organ or tissue. In one example, for a transplant recipient HLA matched with the donor, the protocol includes administration of cyclophosphamide, thymic irradiation, and anti-thymocyte globulin. In another example, for a transplant recipient HLA mismatched with the donor, the protocol includes administration of cyclophosphamide, anti-CD2 antibody, thymic and bone marrow irradiation, and can be with or without rituximab. In another example, for a transplant recipient HLA matched with the donor, the protocol involves administration of cyclophosphamide, fludarabine, and CD34+ cells. Other approaches for promoting allograft acceptance are known to those of skill in the art.

The Tregs or mixed population of Tregs and NK cells can be administered in addition to or in place of any accepted protocols for promoting allograft acceptance. In certain instances, the administration of immunosuppressive agents is decreased after administration of the Tregs. The dose of the immunosuppressive agent can be decreased by about 10%, 20%, 30%, 40%, 50%, 60%, 70%, 80%, 90%, or 100% after administration of the Tregs or the mixed population of Tregs and NK cells. In some examples, the dose of the immunosuppressive agent is decreased by about 50% following treatment with Tregs or the mixed population of Tregs and NK cells. In another example, the administration of immunosuppressive agents is ceased after administration of the Tregs or the mixed population of Tregs and NK cells.

The Tregs or mixed population of Tregs and NK cells of the invention are also useful for the treatment of autoimmune disorders, such as autism, autism spectrum disorder, rheumatoid arthritis, lupus, focal segmental glomerulonephritis, and membranous nephropathy. Further non-limiting examples of an autoimmune disease or disorder include, but are not limited to, inflammatory arthritis, type 1 diabetes mellitus, multiples sclerosis, psoriasis, inflammatory bowel diseases, and vasculitis, allergic inflammation, such as allergic asthma, atopic dermatitis, contact hypersensitivity, Graves' disease (overactive thyroid), Hashimoto's thyroiditis (underactive thyroid), celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome (CFS), autoimmune Addison's Disease, ankylosing spondylitis, acute disseminated encephalomyelitis, antiphospholipid antibody syndrome, aplastic anemia, idiopathic thrombocytopenic purpura, myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, and fibromyalgia (FM).

### Combination Therapy

The Tregs or mixed population of Tregs and NK cells described herein can be used in combination with other known agents and therapies. Administered “in combination,” as used herein, means that two (or more) different treatments are delivered to the subject during the course of the subject's affliction with the disorder (e.g., disease or condition), e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The Tregs or mixed population of Tregs and NK cells described herein and the at least one additional therapeutic agent can be administered simultaneously, in the same or in separate compositions, or sequentially. For sequential administration, the Tregs or mixed population of Tregs and NK cells can be administered first, and the additional agent can be administered second. Alternatively, the order of administration can be reversed, and the additional agent can be administered first, and the Tregs or mixed population of Tregs and NK cells can be administered second. The Treg or mixed population of Tregs and NK cells cell therapy and/or other therapeutic agents, procedures, or modalities can be administered during periods of active disorder, or during a period of remission or less active disease. The Treg or mixed population of Tregs and NK cells cell therapy can be administered before another treatment, concurrently with the treatment, post-treatment, or during remission of the disorder.

When administered in combination, the Tregs or mixed population of Tregs and NK cells described herein and the additional agent (e.g., second or third agent), or all, can be administered in an amount or dose that is higher, lower, or the same as the amount or dosage of each agent used individually, e.g., as a monotherapy. In certain embodiments, the administered amount or dosage of the Tregs or mixed population of Tregs and NK cells, the additional agent (e.g., second or third agent), or all, is lower (e.g., at least 20%, at least 30%, at least 40%, or at least 50%) than the amount or dosage of each agent used individually. In other embodiments, the amount or dosage of the Tregs or mixed population of Tregs and NK cells, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect.

For example, the additional therapeutic agent(s) may include one or more immunosuppressive agents commonly given for organ or tissue transplant. The immunosuppressive agent(s) may be an agent that is given immediately after transplantation to prevent acute rejection (e.g., methylprednisolone, atgam, thymoglobulin, basiliximab, or alemtuzemab) or an immunosuppressive agent(s) used for maintenance (e.g., prednisone, a calcineurin inhibitor (e.g., cyclosporine or tacrolimus), mycophenolate mofetil, azathioprine, sirolimus or everolimus). Other immunosuppressive agents given after organ transplantation include CTLA-4 fusion proteins (e.g., belatacept or abatacept), corticosteroids (e.g., methylprednisolone, dexamethasone, or prednisolone), cytotoxic immunosuppressants (e.g., azathioprine, chlorambucil, cyclophosphamide, mercaptopurine, or methotrexate), immunosuppressant antibodies (e.g., antithymocyte globulins, basiliximab, or infliximab), sirolimus derivatives (e.g., everolimus or sirolimus), and anti-proliferative agents (e.g., mycophenolate mofetil, mycophenolate sodium, or azathioprine). Further immunosuppressants suitable for use the invention described herein are known to those of skill in the art, and the invention is not limited in this respect.

Furthermore, in view of the results described herein, the suppressive activity of donor peptide driven T-cell lines generated from kidney transplant patients involves the adenosinergic pathway. Given these results, an additional combination therapy involves combining infusion of Tregs with additional treatments such as adenosine receptor agonists (e.g., regadenoson) or increasing CD39 expression (e.g., by administering immunomodulatory treatments such as interferon β, fingolimod, alemtuzumab and corticoids) in a graft to achieve transplantation tolerance.

### Administration Routes

An effective amount of a therapeutic agent (e.g., a Treg, or a mixed population of Tregs and NK cells, specific to a donor alloantigen or an autoantigen) described herein for treatment or prevention of a disease or disorder (e.g., transplant rejection or an autoimmune disorder), or for promoting allograft acceptance, can be administered to a subject by standard methods. For example, the agent can be administered by any of a number of different routes including, e.g., intravenous, intraperitoneal, intramuscular, intradermal, subcutaneous, percutaneous injection, oral, transdermal (topical), transarterial, intratumoral, intranodal, intramedullar, or transmucosal. In some embodiments, the agent (e.g., a Treg, or a mixed population of Tregs and NK cells, specific to a donor alloantigen or an autoantigen) can be administered (e.g., by injection or infusion) directly into a transplanted organ or tissue. In one embodiment, the compositions described herein are administered into a body cavity or body fluid (e.g., ascites, pleural fluid, peritoneal fluid, or cerebrospinal fluid). For example, the therapeutic agent (e.g., a Treg, or a mixed population of Tregs and NK cells, specific to a donor alloantigen or an autoantigen) can be administered by injection or infusion, e.g., intramuscularly, subcutaneously, intraperitoneally, or intravenously. The most suitable route for administration in any given case will depend on the particular agent administered, the patient, the particular disease or condition being treated, pharmaceutical formulation methods, administration methods (e.g., administration time and administration route), the patient's age, body weight, sex, severity of the diseases being treated, the patient's diet, and the patient's excretion rate. The agent (e.g., a Treg, or a mixed population of Tregs and NK cells, specific to a donor alloantigen or an autoantigen) can be encapsulated or injected, e.g., in a viscous form, for delivery to a chosen site. The agent can be provided in a matrix capable of delivering the agent to the chosen site. Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, and cosmetic appearance and interface properties. One example is a collagen matrix.

The therapeutic agent (e.g., a Treg, or a mixed population of Tregs and NK cells, specific to a donor alloantigen or an autoantigen) can be incorporated into pharmaceutical compositions suitable for administration to a subject, e.g., a human. Such compositions typically include the agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the described herein. Supplementary active compounds can also be incorporated into the compositions.

### Dosing

The term “unit dosage form” as is used herein refers to a dosage for suitable one administration. By way of example, a unit dosage form can be an amount of therapeutic disposed in a delivery device, e.g., a syringe or intravenous drip bag. For example, a unit dosage form is administered in a single administration. In another example, more than one unit dosage form can be administered simultaneously.

In some embodiments, the Tregs or mixed population of Tregs and NK cells are administered as a monotherapy, i.e., another treatment for the condition is not concurrently administered to the subject. The Treg or mixed population of Tregs and NK cell compositions can be administered once to the patient. If necessary, the Treg cell compositions can also be administered multiple times. The Tregs or mixed population of Tregs and NK cells can be administered by using infusion techniques that are commonly known in immunotherapy (see, e.g., Rosenberg et al., New England Journal of Medicine. 319:1676 (1988)).

The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In other embodiments, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Efficacy

The efficacy of treatment with Tregs or a mixed population of Tregs and NK cells in, e.g., the treatment of transplant rejection or an autoimmune disorder, or the promotion of allograft acceptance, can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate. Treatment according to the methods described herein can reduce levels of a marker or symptom of a condition, e.g., by at least 10%, at least 15%, at least 20%, at least 25%, at least 30%, at least 40%, at least 50%, at least 60%, at least 70%, at least 80%, or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein.

Treatment includes any treatment of a disease in an individual and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein. When using an experimental animal model, efficacy of treatment is evidenced when a statistically significant change in a marker is observed.

Exemplary, non-limiting symptoms of transplant rejection include increase in serum creatinine, decrease in eGFR (estimated glomerular filtration rate), flu-like symptoms, fever, decreased urine output, weight gain, pain, and fatigue.

Exemplary, non-limiting symptoms of an autoimmune disease or disorder include fatigue, joint pain and swelling, skin problems, abdominal pain or digestive issues, recurring fever, proteinuria and swollen glands.
All such modifications are intended to be included within the scope of the appended claims.

The invention is further described in the following numbered paragraphs:

1. An isolated regulatory T cell (Treg) comprising a T cell receptor (TCR) that specifically binds to:

(i) an alloantigen that is a human leukocyte antigen (HLA) molecule, or a fragment thereof, and is not encoded by a nucleotide sequence present in the genome of the Treg, or

(ii) an autoantigen contributing to an autoimmune disorder, or a fragment thereof.

2. The Treg of paragraph 1, wherein the TCR specifically binds to the HLA molecule.

3. The Treg of paragraph 2, wherein the TCR specifically binds to a hypervariable region (HVR) of the HLA molecule.

4. The Treg of paragraph 3, wherein the TCR specifically binds to a β-chain HVR of the HLA molecule.

5. The Treg of any one of paragraphs 2-4, wherein the HLA molecule is an HLA-DR, HLA-DQ, HLA-DP, HLA-A, HLA-B, or HLA-C molecule, or a fragment thereof.

6. The Treg of paragraph 5, wherein the HLA molecule is an HLA-DR, HLA-DQ, or HLA-DP molecule, or a fragment thereof.

7. The Treg of paragraph 6, wherein the HLA-DR molecule is an HLA-DR1, HLA-DR2, HLA-DR3, HLA-DR4, HLA-DR5, HLA-DR6, HLA-DR7, HLA-DR8, HLA-DR9, HLA-DR10, HLA-DR11, HLA-DR12, HLA-DR13, HLA-DR14, HLA-DR15, HLA-DR16, HLA-DR17, HLA-DR18, HLA-DR51, HLA-DR52, or HLA-DR53 molecule, or a fragment thereof.

8. The Treg of any one of paragraphs 1-7, wherein the Treg is capable of suppressing T effector cell (Teff) responses directed towards the alloantigen or the autoantigen.

9. The Treg of paragraph 8, wherein the Treg is capable of suppressing Teff proliferation responses to direct allorecognition, semi-direct allorecognition, and/or indirect allorecognition.

10. The Treg of paragraph 8 or 9, wherein the Treg is capable of activating the adenosinergic signaling pathway.

11. The Treg of any one of paragraphs 1-10, wherein the Treg expresses one or more markers selected from the group consisting of CD4, CD25, CD39, CD73, FOXP3, GITR, CLTA4, ICOS, GARP, LAP, PD-1, CCR6, and CXCR3.

12. The Treg of any one of paragraphs 2-11, wherein the HLA molecule, or the fragment thereof, to which the TCR specifically binds is encoded by a nucleotide sequence that is present in the genome of a donor of an organ or tissue.

13. An isolated Treg comprising a TCR that specifically binds to:

(i) an alloantigen that is an HLA molecule, or a fragment thereof, and is not encoded by a nucleotide sequence present in the genome of the Treg, or

(ii) an autoantigen contributing to an autoimmune disorder, or a fragment thereof;

wherein the Treg has been produced by a method comprising:
(a) contacting an immune cell population comprising T cells obtained from a recipient subject with a fragment of the HLA molecule or autoantigen and an autologous antigen-presenting cell (APC); and
(b) expanding the immune cell population of step (a) for a time and under conditions sufficient to form an expanded T cell line comprising a plurality of the Tregs; and, optionally
(c) purifying the Tregs from the immune cell population.

14. The Treg of paragraph 13, wherein the immune cell population of (a) further comprises natural killer (NK) cells, and, if step (c) is performed, step (c) comprises purifying the Tregs and NK cells from the immune cell population, thereby producing a mixed population of Tregs and NK cells.

15. A mixed population of cells comprising the Treg of any one of paragraphs 1-12 and NK cells.

16. A composition comprising the Treg of any one of paragraphs 1-14.

17. A composition comprising the mixed population of cells of paragraph 15.

18. A method of suppressing an immune response in a subject, the method comprising administering the Treg of any one of paragraphs 1-14, the mixed population of cells of paragraph 15, or the pharmaceutical composition of paragraph 16 or 17, to the subject.

19. The method of paragraph 18, wherein the immune response is a Teff response directed towards the alloantigen or the autoantigen.

20. A method of treating or preventing transplant rejection or a method of treating an autoimmune disorder in a subject, the method comprising administering the Treg of any one of paragraphs 1-14, the mixed population of cells of paragraph 15, or the composition of paragraph 16 or 17, to the subject.

21. The method of paragraph 18 or 19, wherein the subject has an autoimmune disorder.

22. The method of any one of paragraphs 18-20, wherein the subject is an organ or tissue transplant recipient.

23. The method of any one of paragraphs 18-20 or 22, wherein the HLA molecule, or the fragment thereof, to which the TCR specifically binds is encoded by a nucleotide sequence that is present in the genome of the donor of the organ or tissue.

24. The method of any one of paragraphs 18-20, 22, or 23, wherein the method further comprises reducing the dose of an immunosuppressive agent administered to the subject.

25. The method of any one of paragraphs 18-20 or 22-24, wherein the organ is a kidney, a liver, a heart, a lung, a pancreas, an intestine, a stomach, a testis, a penis, a thymus, or a face, hand, or leg vascular composite allograft.

26. The method of any one of paragraphs 18-20 or 22-24, wherein the tissue comprises bone, a tendon, a cornea, skin, a heart valve, nervous tissue, bone marrow, islets of Langerhans, stem cells, blood, or a blood vessel.

27. The method of paragraph 20 or 21, wherein the autoimmune disorder is autism, autism spectrum disorder, rheumatoid arthritis, lupus, focal segmental glomerulonephritis, or membranous nephropathy.

28. A method for producing the Treg of any one of paragraphs 1-12, the method comprising:

(a) contacting an immune cell population comprising T cells obtained from a recipient subject with a fragment of the HLA molecule or autoantigen and an autologous APC; and

(b) expanding the immune cell population of step (a) for a time and under conditions sufficient to form an expanded T cell line comprising a plurality of the Tregs; and, optionally

(c) purifying the Tregs from the immune cell population.

29. The method of paragraph 28, wherein the method comprises repeating steps (a) and (b) more than three times.

30. The method of paragraph 28 or 29, wherein the method comprises repeating steps (a) and (b) four or five times.

31. The method of any one of paragraphs 28-30, wherein step (a) is performed about every seven to ten days.

32. The method of any one of paragraphs 28-31, wherein the autologous APCs are peripheral blood mononuclear cells (PMBCs), dendritic cells, macrophages, or B cells.

33. The method of paragraph 32, wherein the autologous APCs are PBMCs.

34. The method of paragraph 33, wherein the PBMCs are irradiated.

35. The method of any one of paragraphs 28-34, wherein the immune cell population comprising T cells is a population of PMBCs, a population of naïve T cells, or a population of purified Tregs.

36. The method of paragraph 35, wherein the immune cell population is a population of PBMCs.

37. The method of paragraph 36, wherein step (a) further comprises contacting the population of PBMCs with IL-2.

38. The method of paragraph 37, wherein the concentration of IL-2 is about 50 IU/ml to about 200 IU/ml.

39. The method of paragraph 38, wherein the concentration of IL-2 is about 100 IU/ml.

40. The method of any one of paragraphs 28-39, wherein the concentration of the fragment of the HLA molecule or autoantigen is about 25 μg/ml to about 200 μg/ml.

41. The method of paragraph 40, wherein the concentration of the fragment of the HLA molecule or autoantigen is about 50 μg/ml.

42. The method of any one of paragraphs 28-41, wherein the fragment of the HLA molecule or autoantigen is a purified peptide or peptide mixture.

43. The method of any one of paragraphs 28-42, wherein the immune cell population comprises NK cells.

44. The method of any one of paragraphs 28-43, wherein step (c) comprises purifying the Tregs and NK cells from the immune cell population, thereby producing a mixed population of Tregs and NK cells.

45. A composition comprising:

(a) the Treg of any one of paragraphs 1-14; and

(b) a fragment of the HLA molecule or autoantigen.

46. The composition of paragraph 45, wherein the composition further comprises IL-2.

47. The composition of paragraph 46, wherein the concentration of IL-2 is about 50 IU/ml to about 200 IU/ml.

48. The composition of paragraph 47, wherein the concentration of IL-2 is about 100 IU/ml.

49. The composition of any one of paragraphs 45-48, wherein the concentration of the fragment of the HLA molecule or autoantigen is about 25 μg/ml to about 200 μg/ml.

50. The composition of paragraph 49, wherein the concentration of the fragment of the HLA molecule or autoantigen is about 50 μg/ml.

51. The composition of any one of paragraphs 45-50, wherein the fragment of the HLA molecule or autoantigen is a purified peptide or peptide mixture.

52. The composition of any one of paragraphs 45-51, further comprising NK cells.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description

The technology described herein is further illustrated by the following examples which in no way should be construed as being further limiting.

### EXAMPLES

The following are examples of the methods and compositions of the invention. It is understood that various other embodiments may be practiced, given the description provided herein.

### Example 1. Patients for Study

A total of 45 kidney transplant recipients with one or more HLA-DR mismatches with the donor were included in the study. Patients were treated with double or triple immunosuppressive therapy including tacrolimus, except in three cases where the patient received everolimus or belatacept instead of tacrolimus. Blood samples were obtained at various post-transplant visits after obtaining informed consent and nineteen T cell lines were generated from seventeen patients. The local institutional ethics committee approved the study protocol.

### Example 2. Generation of HLA-DR-Specific T Cell Lines

### Synthesis of Peptides

A panel of non-overlapping peptides 18-22 amino acids in length was synthesized corresponding to the full-length β-chain hypervariable regions of HLA-DR81*0101, HLA-DR81*1501, HLA-DR81*0301 and HLA-DR81*0401 (PROIMMUNE®, Littlemore, UK), as previously reported (Tsaur et al., Kidney Int. 79(9):1005-12, 2011).

### Generation of T Cell Lines

Peripheral blood samples of kidney transplant recipients were collected at various visits post transplantation and peripheral blood mononuclear cells (PBMCs) were isolated by density gradient centrifugation using LYMPHOPREP™ (Stemcell Technologies). The cells were then expanded ex vivo or frozen in LN2 for future use.

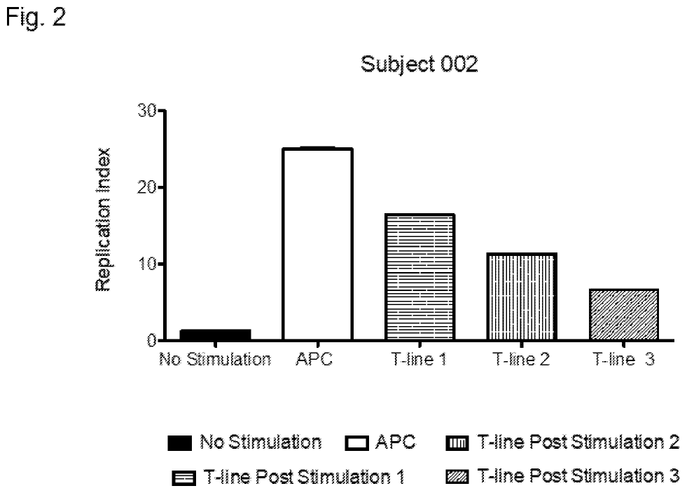

PBMCs (10×10⁶) were cultured in IMMUNOCULT™ serum-free culture medium (Stemcell Technologies), containing 100 U/mL penicillin, 100 μg/mL streptomycin, 100 μg/mL L-glutamine, 5 mmol/L HEPES, 1% nonessential amino acids, and 1 mmol/L sodium pyruvate (Gibco), and 2-mercaptoethanol. The PBMCs were repeatedly stimulated at 7-10 day intervals with the mismatched donor-derived HLA-DR allopeptides (50 μg/mL) and autologous irradiated (10-15 Gy) PBMC as antigen-presenting cells (APC) in the presence of IL-2 (10 μg/mL) as described in Tsaur et al., Kidney Int. 79(9):1005-12 (2011). All T cell lines were cultured at 37° C. in a humidified 5% CO₂ incubator and harvested after four to five cycles of stimulation. The immunoregulatory function of the expanded cells was assessed by a suppression assay and was shown to be capable of suppressing CD4⁺ T cell proliferation in response to the same donor antigen (FIG. 2).

### Example 3. Proliferation and Suppression Assay

1×10⁶ carboxyfluorescein succinimidyl ester (CFSE) stained PBMCs were used as responders and stimulated with donor-mismatched HLA-DR allopeptide and autologous irradiated PBMCs as APCs (2×10⁶) for 72 h in a humidified 5% CO₂ incubator in a 96 well U bottom plate. Proliferation was assessed by dilution of CFSE. Stimulated PBMCs were cultured in presence or absence of T cell lines at a PBMC:T cell line ratio ranging from 1:2 to 1:16 in the suppression assays.

For contact-independent suppression assays, a transwell plate was used instead of a 96 well U bottom plate. Experiments involving inhibition of suppression included addition of istradefynille (20 μg/mL) or anti-IL-10 (10 μg/mL) and anti-TGF-β (10 μg/mL) neutralizing antibodies. All assays were performed in triplicate.

### Example 4. Flow Cytometric Analysis

T cell lines were immunophenotyped for various T cell markers with fluorophore-conjugated human anti-CD3, anti-CD4, anti-CD25, anti-CD127, anti-CD39, and anti-CD73 (BIOLEGEND®). The data were acquired using a Canto II cytometer (BD BIOSCIENCES®) and analyzed using FLOWJO®. The gating strategy for phenotyping included initial gating of live PBMC population followed by the CD3⁺CD4⁺ population. The expression levels of CD25, CD127, CD39, and CD73 were expressed as % of the CD3⁺CD4⁺ population.

T cell proliferation and suppression was determined by CFSE dye dilution of the responder cells. Analysis of CFSE distribution was performed on FLOWJO® Proliferation platform and data are represented by Replication Index (RI). RI determines the fold-expansion of only the responding cells (Roederer, Cytometry A. 79(2):95-101 (2011)) and is defined as the average number of divisions that all cells undergo after they are stained by a cell proliferation dye. The percentage of suppression was calculated from the proliferation and suppression values.

### Example 5. Statistical Analysis

Results are expressed as mean±s.d. Characteristics of patients, phenotype, and functional data were compared using Student's t-test as appropriate. Each experimental condition was repeated three times. A p<0.05 was considered significant.

### Example 6. Proliferative Response of CD4+CD25− T Cells from Transplant Recipients in Response to Donor-Specific Alloantigen

CD4⁺CD25⁺ cells were depleted from the PBMCs of kidney transplant recipients. The CD4⁺CD25⁻ cells (i.e., Treg-depleted T cell pool) and the CD4⁺ cells were stimulated with the donor alloantigen. Proliferation was measured using a dye dilution method in a flow cytometer.

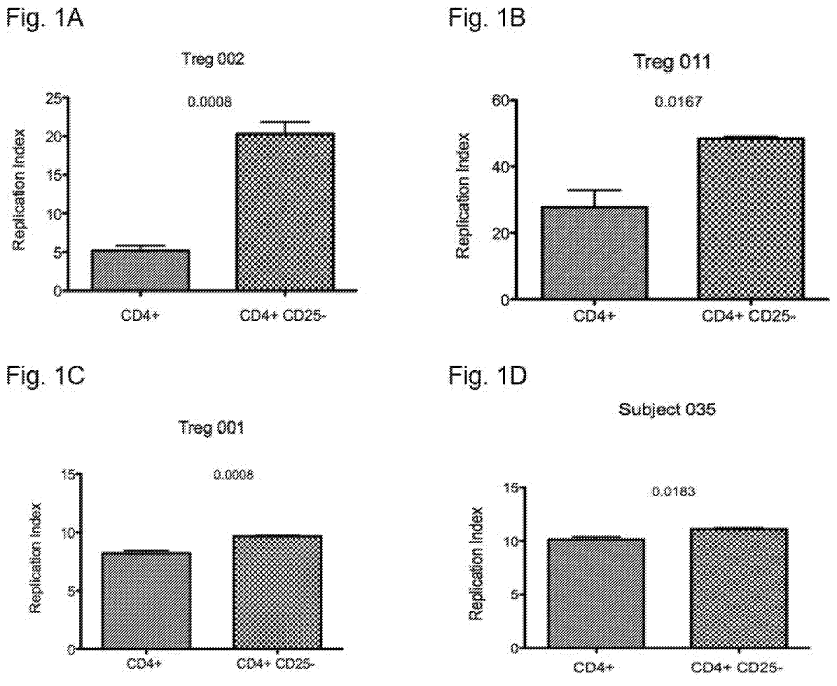

The depletion of Tregs from the T cell pool resulted in an enhanced Teff response towards the alloantigen. This response differs from one recipient to the other (FIGS. 1A-1D).

### Example 7. Immunosuppressive Activity of Generated T Cell Lines

### Demographic and Clinical Characteristics

The study included a total of 45 kidney transplant recipients with one or more donor DR mismatches. A total of 19 T cell individual lines were created and expanded ex vivo from peripheral blood mononuclear cells (PBMC) of 17 subjects. The demographic data of these 17 subjects are presented below in Table 3.

TABLE 3
Demographic data of patients from whom T cell lines were generated

| | n = 17 |
|---|---|
| Age (years) | 51.6 ± (16) |
| Sex | |
| Female (%) | 47 |
| Male (%) | 53 |
| HLA mismatch (number) | 4.5 ± (1.3) |
| Serum creatinine (mg/dL) | 1.5 ± (0.4) |
| Proteinuria > 0.5 g/24 h | 2 |
| Acute/chronic rejection (total number) | 4/1 |
| Type of donor | |
| Deceased (%) | 47 |
| Living (%) | 53 |
| Time (months) between date of | 17 ± (21.5) |
| transplant and C1 | |

HLA—human lymphocyte antigen; C1—the first blood collection; values for age, HLA mismatch, serum creatinine, and time between date of transplant and C1 are expressed as mean ± (s.d.).

In all, 82.35% of the patients received thymoglobulin as induction therapy. With respect to maintenance immunosuppressive treatments, fourteen total patients (82.3%) were in treatment with tacrolimus, ten of whom were in combination with mycophenolate mofetil (MMF) or mycophenolic acid (MPA) and steroids. One patient received everolimus with MMF, while another two patients received belatacept and steroids, one of whom was also treated with MMF, and the other azathioprine. All patients had stable renal function, although four of them had had a previous episode of treated acute rejection.

### Generation and Morphological Characterization of T Cell Lines

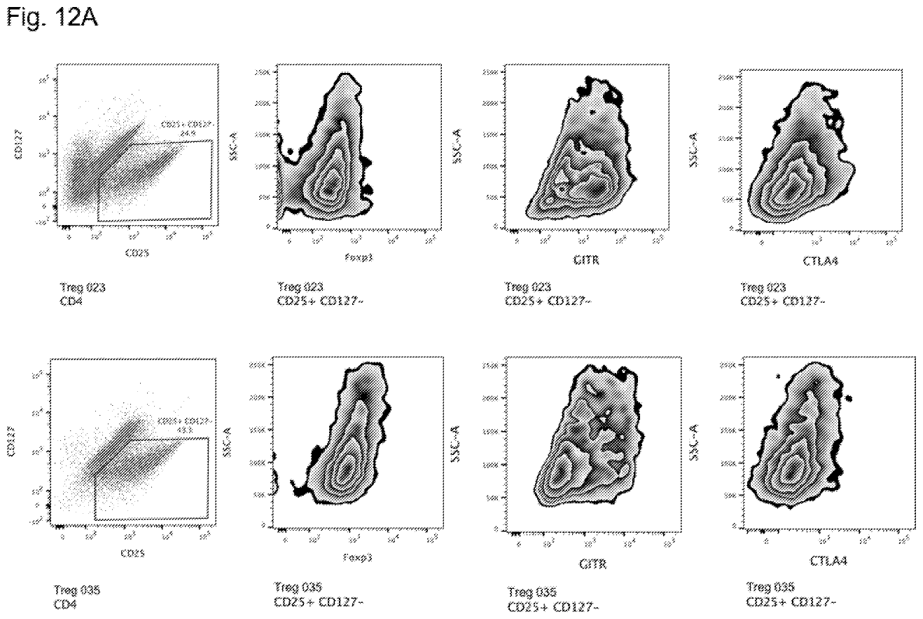

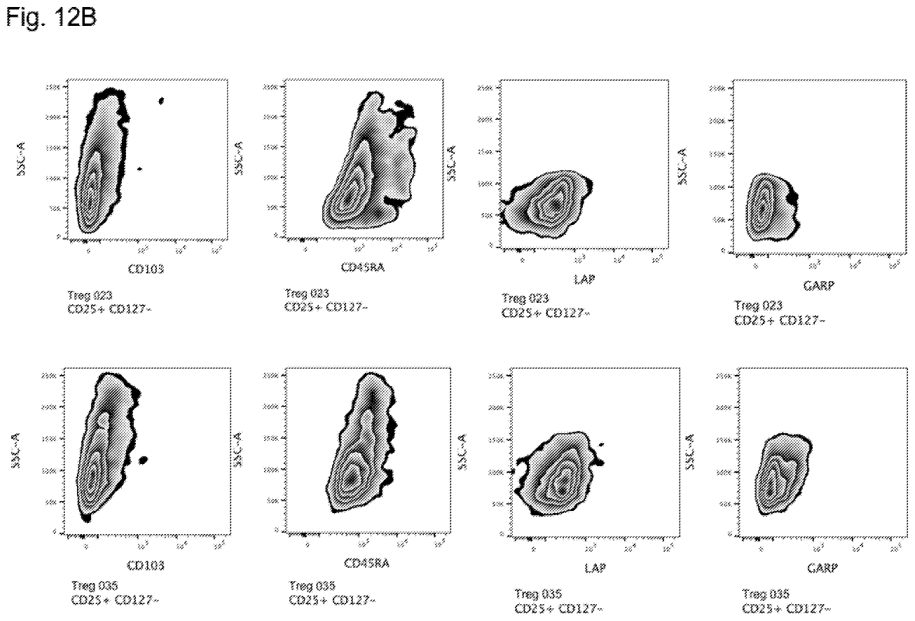

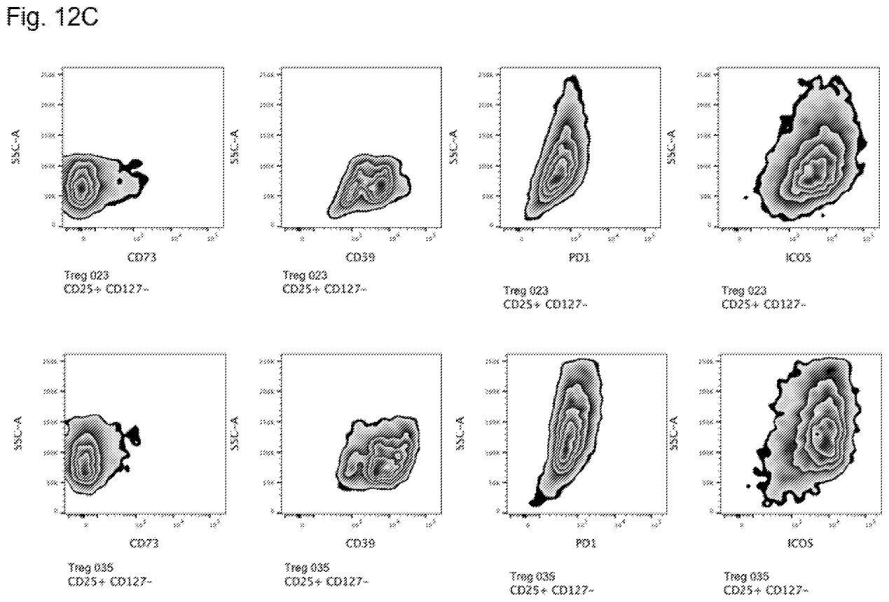

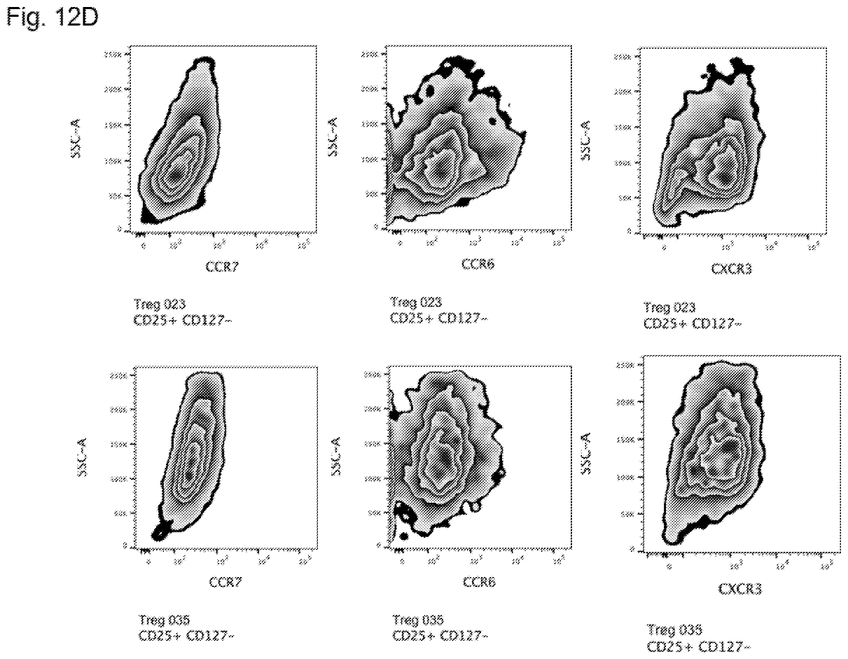

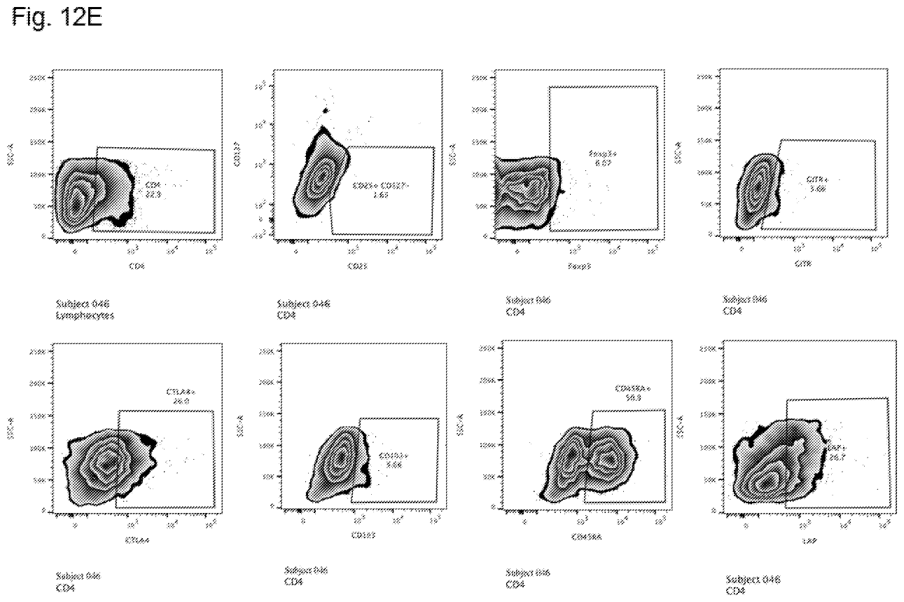

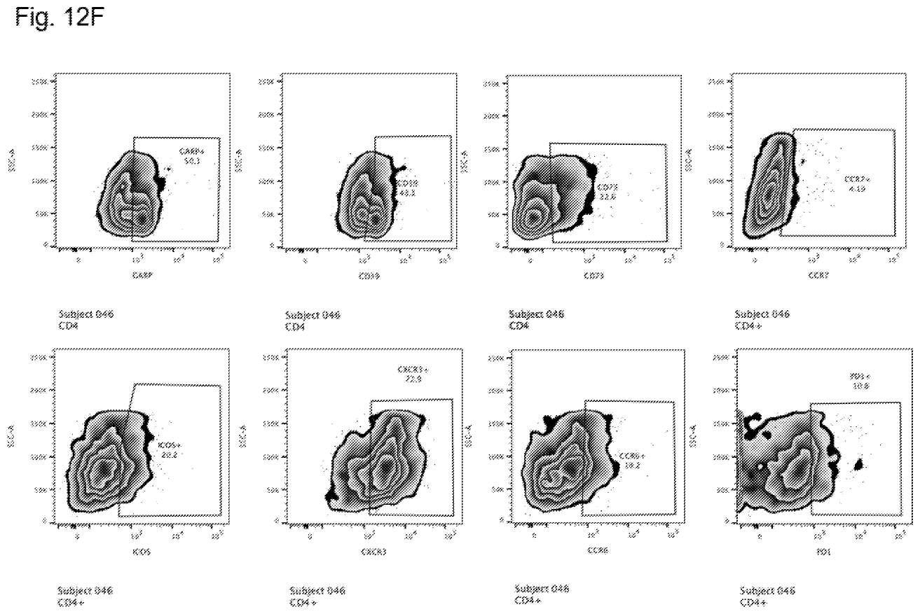

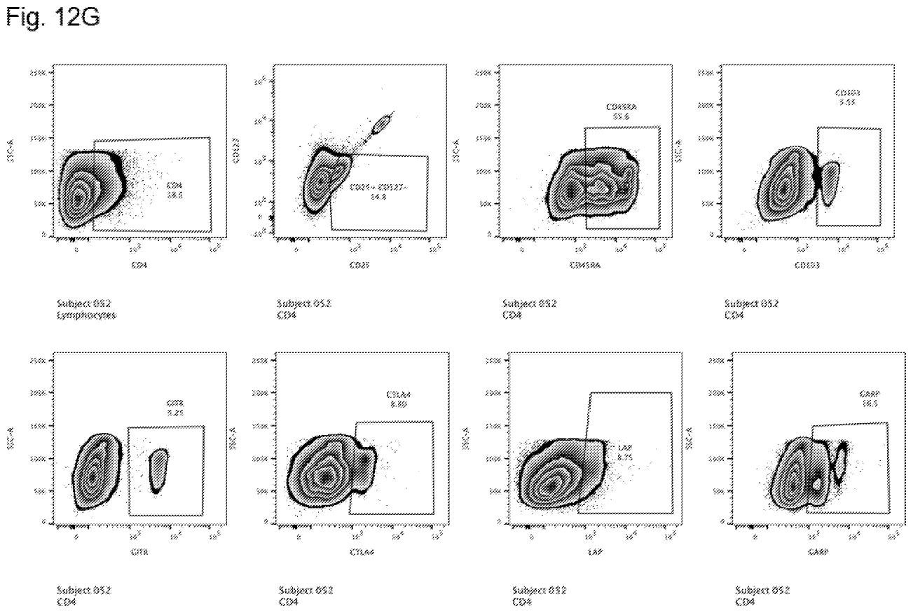

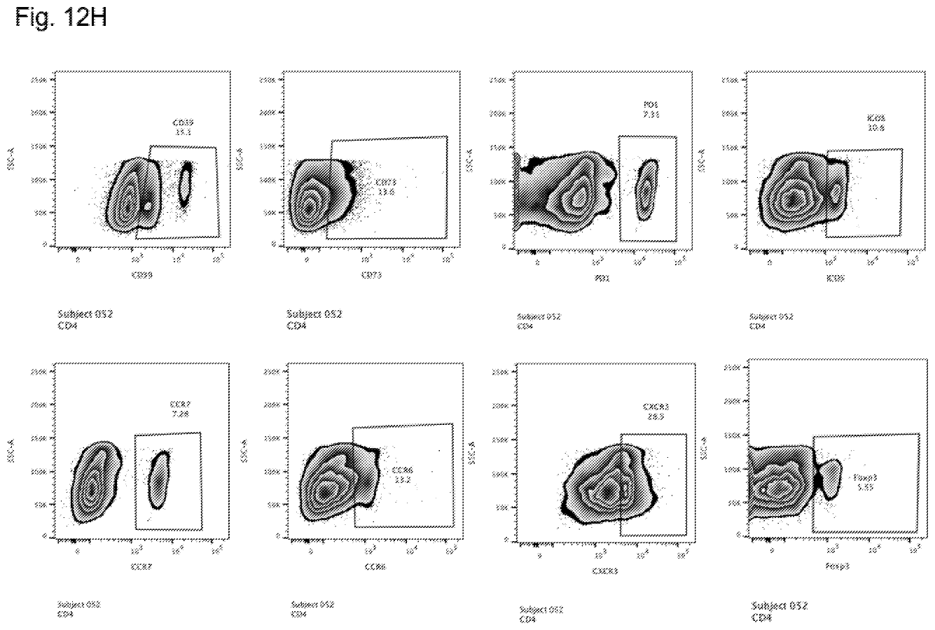

The T cell lines were generated by repeated stimulations (4-5 times) of PBMC from the kidney transplant recipients with donor specific HLA-DR allopeptides (HLA-DR1, HLA-DR4, HLA-DR15, or HLA-DR17) as described in Example 2. Each T cell line generated was analyzed for cell surface markers to define the ex vivo expanded cells. It was observed that 20-50% of cells in the ex vivo expanded lines were CD3⁺CD4⁺ T cells. Some of the generated CD3⁺CD4⁺ T cells also upregulated expression of CD25 simultaneously downregulating CD127 expression. In addition, consistent expression of CD39 and CD73 by the CD3⁺CD4⁺ T cells was observed. The percentage of CD4⁺ T cells, CD4⁺CD25⁺CD125⁻, CD4⁺CD39⁺, and CD4⁺CD73⁺ cells from each ex vivo expanded T cell lines is shown in Table 4. Flow cytometry data of four representative T cell lines are presented in FIGS. 12A-12H.

In all the ex vivo expanded T cell lines, a subset of the CD4⁺ T cells expressed a regulatory phenotype (CD25⁺CD127⁻CD39⁺ and CD25⁺CD127⁻CD73⁺). The percentage of cells expressing CD4⁺CD39⁺ and CD4⁺CD73⁺ varied from 20% to 60%. It was further observed that CD39 and CD73 were not co-expressed on the same CD4⁺ T cell population.

TABLE 4
Phenotyping of generated T cell lines

| | HLA | CD3⁺CD4⁺ | CD4⁺CD25⁺CD127⁻ | CD4⁺CD39⁺ | CD4⁺CD73⁺ |
|---|---|---|---|---|---|
| Patient | mismatch | (%) | (%) | (%) | (%) |
| 002 | DR1 | 52.9 | 14.2 | 16.6 | 30.3 |
| 005 | DR4 / DR17 | 16.5 / 13.6 | 13.5 / 3.95 | 25 / 15.9 | 48.7 / 17.8 |
| 008 | DR4 | 56.3 | 16.6 | 62.3 | 41.5 |
| 016 | DR4 | 28.1 | 17.3 | 47.8 | 48.8 |
| 018 | DR4 | 40.2 | 24.8 | 63.8 | 10.1 |
| 022 | DR15 | 41.6 | 9.71 | 19 | 56.2 |
| 023 | DR15 | 32.2 | 27.5 | 26.4 | 23.3 |
| 037 | DR4 | 58.9 | 7.1 | 61.8 | 32.8 |
| 046 | DR4 | 20.8 | 10.6 | 24.8 | 17.4 |
| 048 | DR15 | 25.1 | 3.04 | 38 | 25.3 |
| 004 | DR15 | 32.4 | 17.6 | 57.4 | 14.2 |
| 011 | DR4 | 16.6 | 20.4 | 24.5 | 32.6 |
| 035 | DR1 | 49.9 | 48.6 | 67.7 | 24.3 |
| 036 | DR1 / DR15 | 13.4 / 18.9 | 9.59 / 14.4 | 19.2 / 31 | 36.3 / 31.7 |
| 001 | DR4 | 39.5 | 36.1 | 55 | 44.3 |
| 038 | DR4 | 25.3 | 20 | 18.8 | 22.4 |
| 039 | DR1 | 36.2 | 23.8 | 41.8 | 29.5 |

### Functional Characterization of T Cell Lines

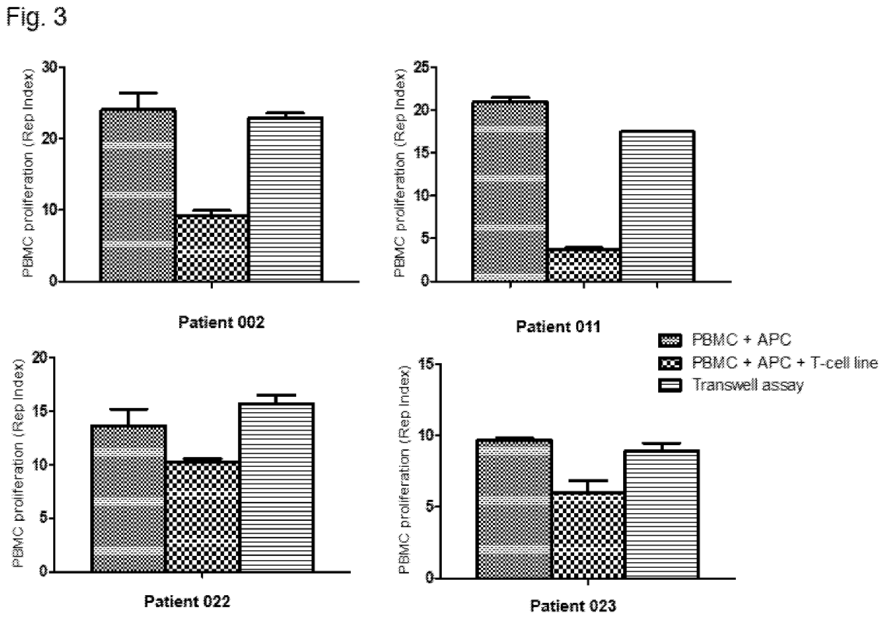

The functional characterization of the ex vivo expanded T cell lines was next determined by assessing their immunosuppressive function to inhibit antigen specific and non-specific T cell proliferation. It was observed that all 19 T cell lines were able to inhibit donor antigen specific T cell proliferation. The proliferative response of the recipient PBMCs to donor specific HLA-DR allopeptide and the immunosuppressive ability of the T cell lines are presented in Table 5. All the ex vivo expanded T cell lines generated from the 17 transplant recipients demonstrated suppressive ability independent of the different combinations of immunosuppressive drug regimen received by the subjects (Table 5, FIG. 3). The T cell lines did not suppress a non-specific T cell proliferation.

The data shown below in Table 5 is presented using the replication index, which is defined as the average number of divisions that all cells undergo after they are stained by a cell proliferation dye. The percentage of suppression is calculated from proliferation and suppression values (Roederer, Cytometry A. 79(2):95-101 (2011)).

TABLE 5
Proliferative response from PBMC and suppressive capacity of T cell lines

| | | HLA | Proliferation | Suppression | |
|---|---|---|---|---|---|
| Patient | Immunosuppression | mismatch | (Rep index) | (Rep index) | % Suppression |
| 002 | Fk + MPA/MMF + | DR1 | 24.06 | 9.19 | 61.80 |
| 005 | steroids | DR4/DR17 | 16.1/15.96 | 11.36/11.56 | 29.44/27.56 |
| 008 | | DR4 | 9.77 | 7.98 | 18.32 |
| 016 | | DR4 | 8.45 | 6.67 | 21.07 |
| 018 | | DR4 | 10.04 | 6.37 | 36.55 |
| 022 | | DR1/DR15 | 13.60 | 10.22 | 24.85 |
| 023 | | DR15 | 7.96 | 4.37 | 45.10 |
| 037 | | DR4 | 6.37 | 5.20 | 18.37 |
| 046 | | DR4 | 10.04 | 4.93 | 50.89 |
| 048 | | DR15 | 9.46 | 6.4 | 32.34 |
| 004 | Fk + MPA/MMF or | DR15/DR17 | 12.73 | 6.39 | 49.80 |
| 011 | Fk + steroids | DR4 | 6.86 | 3.08 | 55.10 |
| 035 | | DR1 | 5.84 | 2.71 | 53.60 |
| 036 | | DR1/DR15 | 12.53/13.73 | 9.85/10.96 | 21.39/20.17 |
| 001 | Eve + MMF | DR4 | 18.13 | 13.6 | 24.99 |
| 038 | Belatacept + | DR4 | 9.24 | 5.18 | 43.94 |
| 039 | MMF/Aza + steroids | DR1 | 6.96 | 3.91 | 43.82 |

Rep index—replication index; Fk—tacrolimus; MMF—mycophenolate mofetil; MPA—mycophenolic acid; Aza—azathioprine; HLA—human lymphocyte antigen

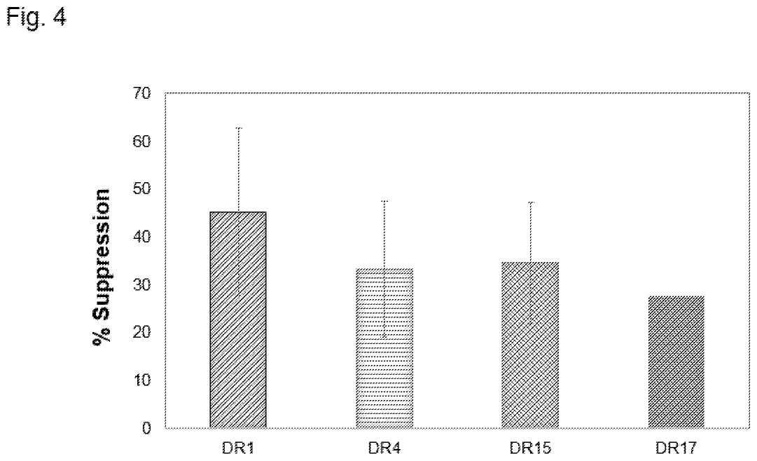

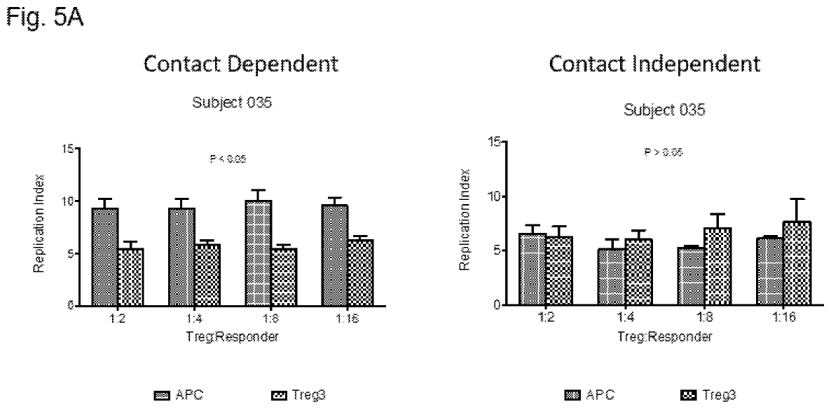

There was no significant difference observed between the specific HLA-DR allopeptide and the percentage of suppression (FIG. 4). Likewise, there was no correlation observed between the percentage of CD4⁺CD25⁺CD127⁻ cells in the generated T cell lines and the percentage of suppression, nor between the percentage of suppression and the renal function of these patients. The difference in percentage of CD4⁺CD25⁺CD127⁻ cells and percentage suppression in the T cell lines between the transplanted patients who were or were not treated for acute rejection was also analyzed. Again, no statistically significant difference was observed.

### Example 8. Contact-Dependent Immunosuppression

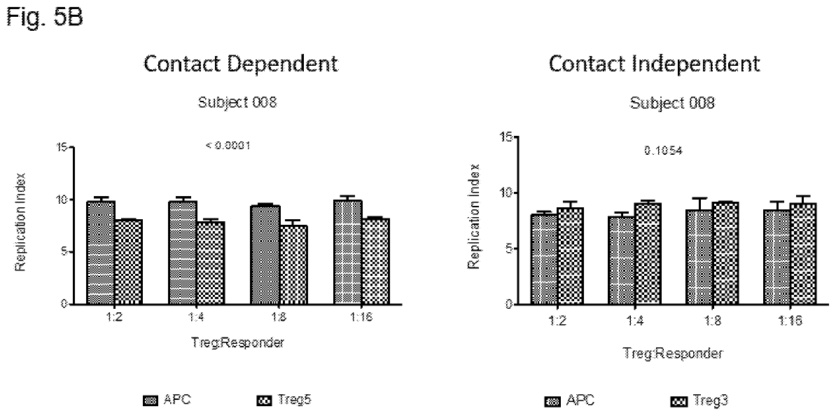

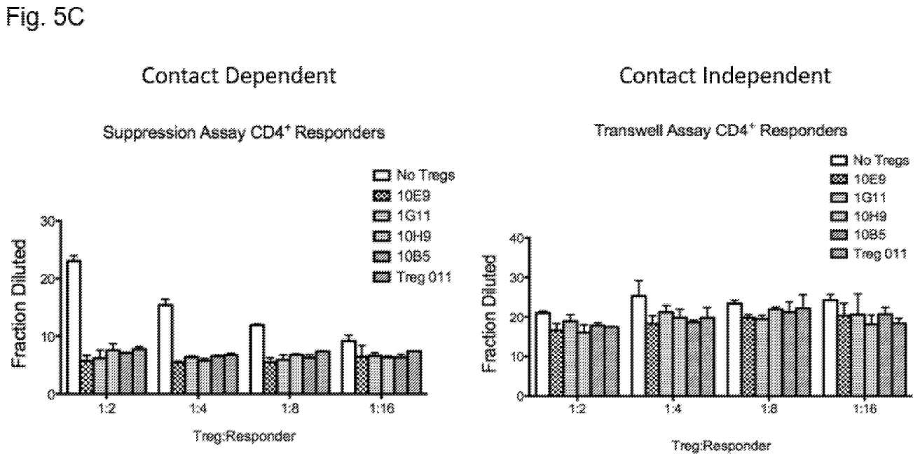

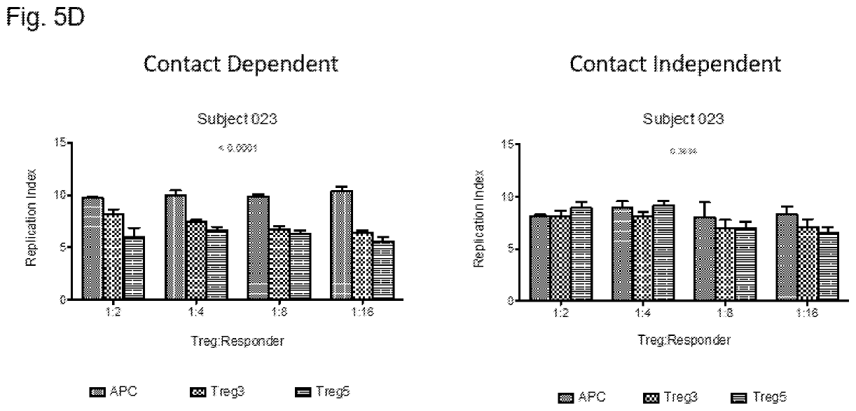

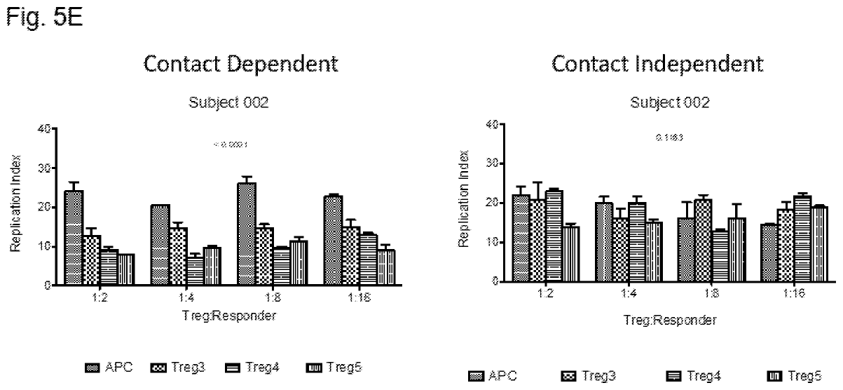

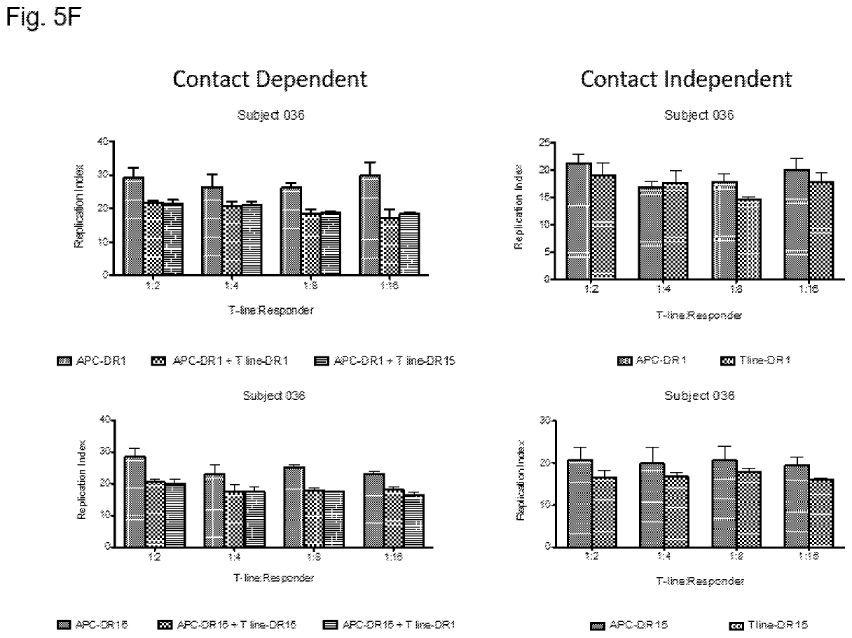

To further understand the mechanism of suppression, a classical transwell system of suppression assay was used as described in Example 3. It was observed that the T cell lines lost their ability to suppress donor antigen-specific T cell proliferation when separated by a semipermeable membrane, demonstrating that the T cell line-mediated suppression is dependent on cell-cell contact (FIGS. 5A-5F).

### Example 9. Suppressive Ability in Response to Direct and Indirect Allorecognition

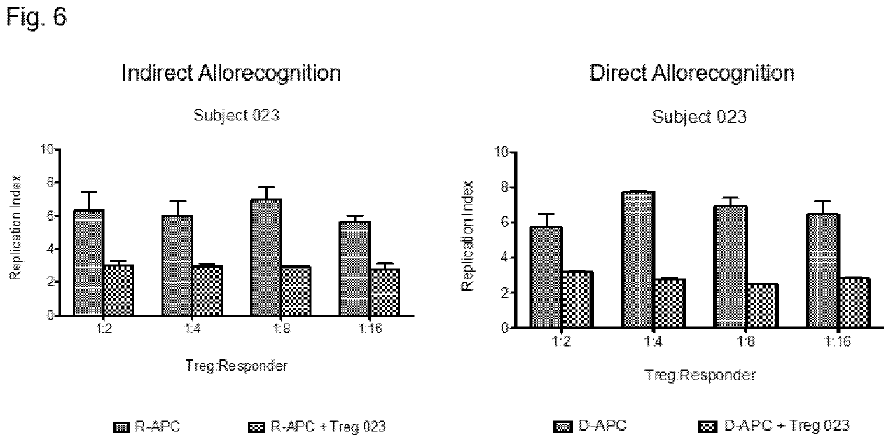

CD4⁺ T cells from kidney transplant recipients were stimulated either by donor cells (direct allorecognition) or autologous APCs loaded with donor antigen (indirect allorecognition) and proliferation/suppression was measured by dye dilution method. The ex vivo expanded immunoregulatory T cell lines effectively suppressed CD4⁺ T cell proliferative response to both direct and indirect allorecognition (FIG. 6). This is desirable in allograft recipients to provide protection against both acute and chronic rejection.

### Example 10. Antigen Specificity of Tregs

Antigen-specific suppression by ex vivo expanded T cell lines were determined using a standard suppression assay. Proliferative response of CD4⁺ T cells from kidney transplant recipients and third party responders to donor antigen was measured by dye dilution method.

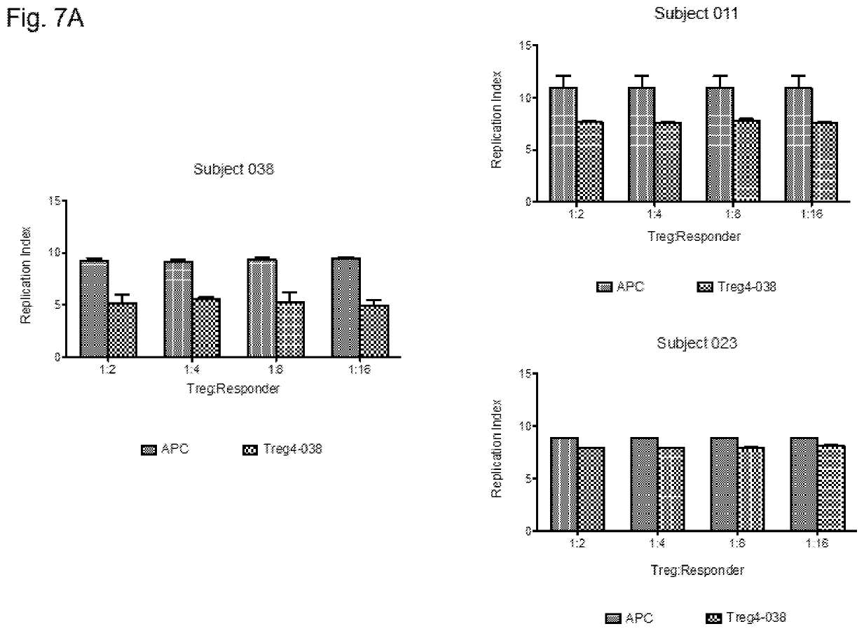

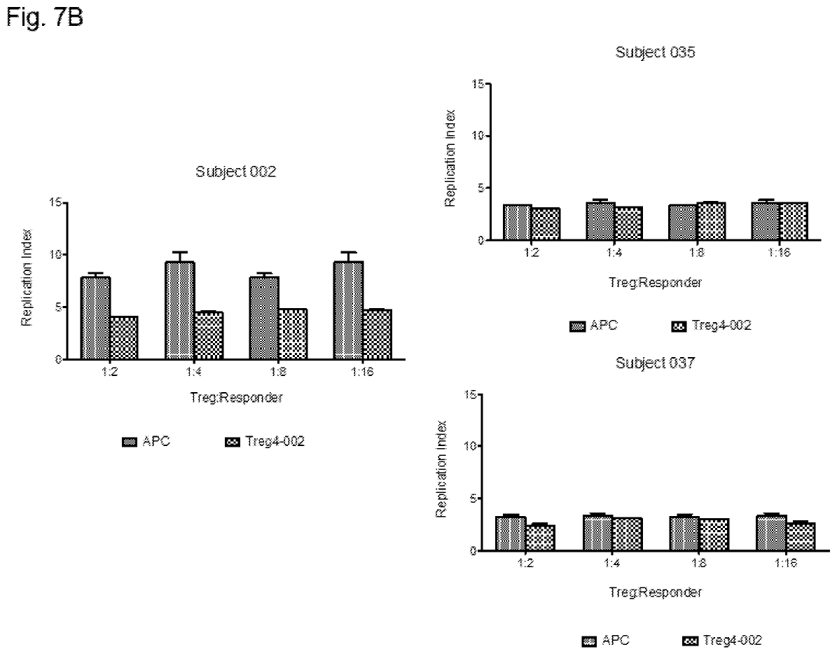

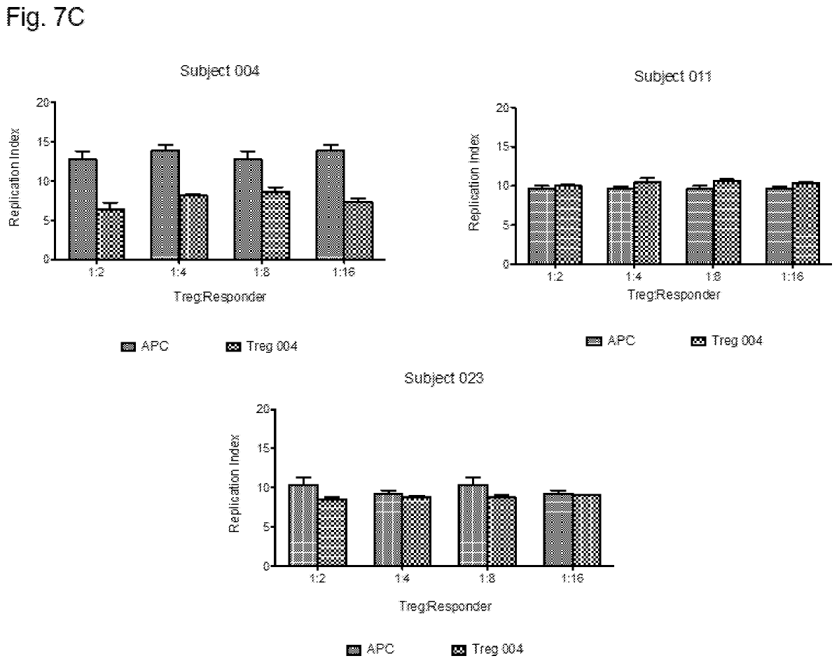

It was observed that the T cell lines selectively suppress T cell proliferative immune response against the specific donor alloantigen, and that the Tregs have no effect on the proliferative response of a third party responder to a different donor antigen. As shown in FIG. 7A, subjects 038 and 023 have a different HLA-DR mismatch with their respective donors (HLA-DR4 mismatch and HLA-DR15 mismatch, respectively). The T line expanded from subject 038 suppressed immune response in said subject against the donor alloantigen, but did not suppress third party immune response against a different alloantigen in subject 023. On the other hand, subjects 038 and 011 have the same HLA-DR mismatch. The T line expanded from subject 038 shows the ability to partially suppress the activation of T responders to the same antigen in a different subject. In FIG. 7B, subjects 002 and 035 have the same HLA-DR mismatch (HLA-DR1), while subject 037 has a different HLA mismatch (HLA-DR4). In FIG. 7C, subject 004, 023, and 011 all have different HLA mismatches (HLA-DR1, -DR15, and -DR4, respectively).

### Example 11. Bystander Suppression Effect

Bystander suppression was determined in subjects with more than one HLA mismatch with their donor. Antigen-specific T cell lines were expanded separately for each mismatch. Bystander suppression effect for each T cell line was determined using a standard suppression assay, where antigenic stimulation as provided by either the same or a different donor peptide.

Although T cells are normally specific to a particular antigen, it was shown in this case that the Tregs were able to suppress immune response to an antigen that was co-expressed along with the antigen to which they are specific, demonstrating an example of linked (bystander) suppression.

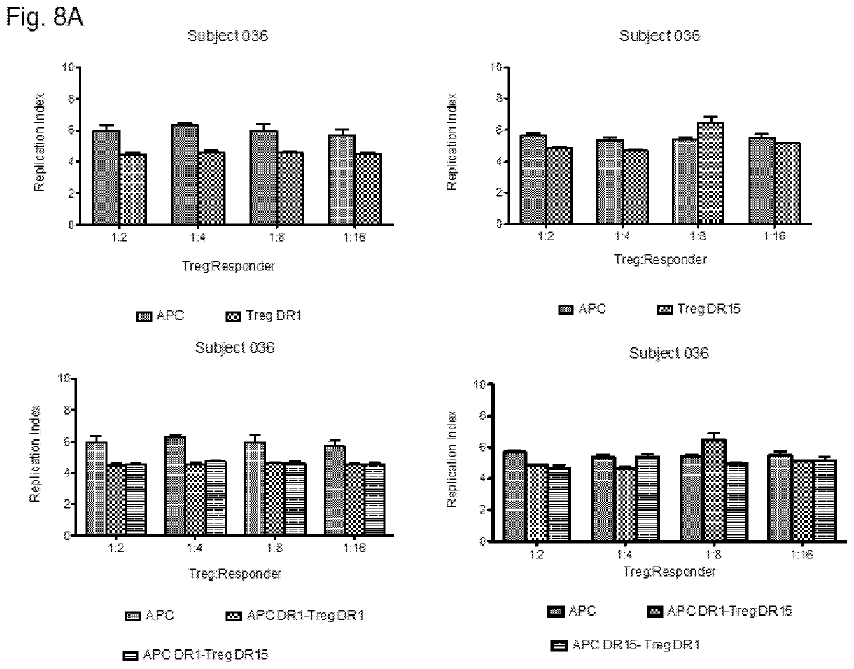

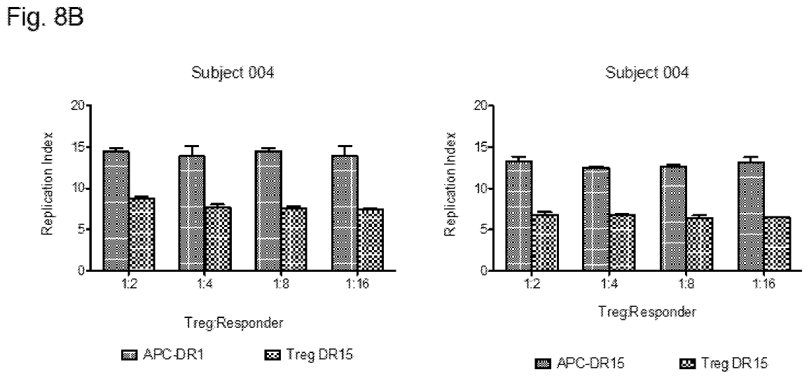

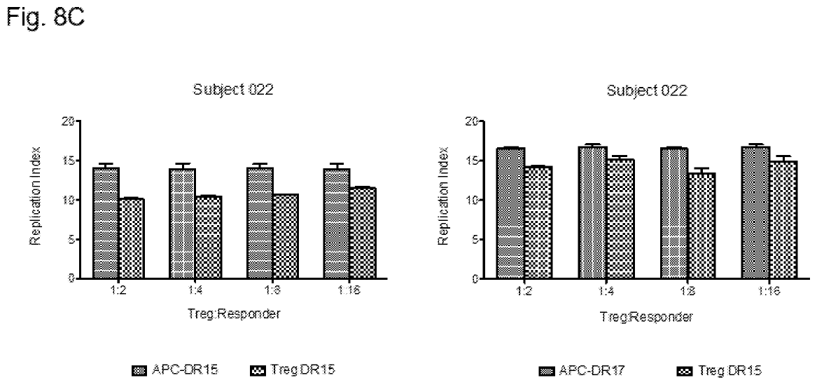

For example, in FIG. 8A, subject 036 had two HLA mismatches, HLA-DR1 and HLA-DR15. As shown in the top two graphs, Tregs against HLA-DR1 and Tregs against HLA-DR15 demonstrated immunosuppressive effect against APCs presenting their respective antigen. In the bottom two graphs, both Treg lines were also capable of suppressing immune response against APCs presenting the co-expressed alloantigen. HLA-DR1 Tregs suppressed immune response against both HLA-DR1 and HLA-DR15; similarly, HLA-DR15 Tregs suppressed immune response against both HLA-DR1 and HLA-DR15. This effect was also demonstrated in FIG. 8B for subject 004, who had HLA-DR1 and HLA-DR15 mismatches, and in FIG. 8C for subject 022, who had HLA-DR15 and HLA-DR17 mismatches.

### Example 12. Mechanisms of Immunosuppression

Upregulation in the expression of both CD39 and CD73 in the generated T cell lines was detected as described above. It has been previously shown that both CD39 and CD73 are implicated in mechanism of immunosuppression in mice Tregs, where the production of ATP-derived adenosine from CD39/CD73-mediated degradation of extracellular ATP to AMP increases the intracellular cyclic AMP (cAMP) level in Tregs. This is transferred to T effector cells through gap junctions, leading to the upregulation of inducible cAMP early repressor (ICER) and, in turn, the inhibition of the nuclear factor of activated T cells (NFAT) and IL-2 transcription (Deaglio et al., J. Exp. Med. 204(6):1257-65, 2007; Klein et al., Front. Immunol. 7:315, 2016).

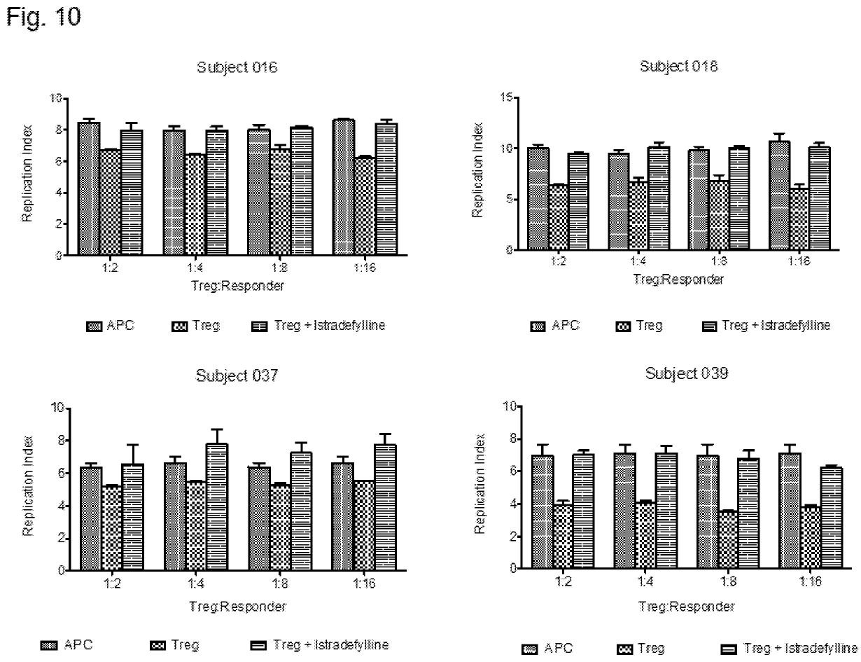

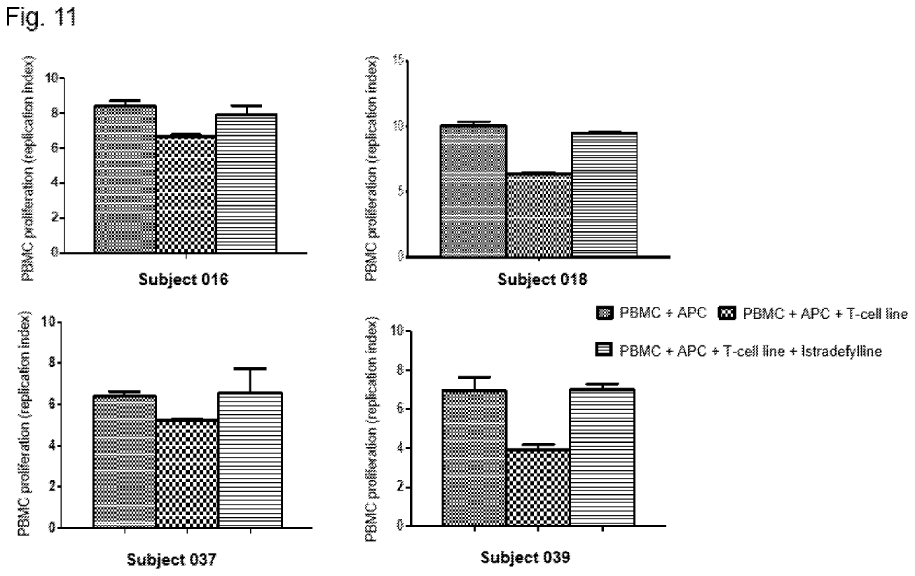

To determine if the mechanism of immunosuppression by the generated T cell lines involves the activation of the adenosinergic pathway similar to mice Tregs, a standard suppression assay in presence or absence of an A2A receptor (A2Ar) antagonist was performed. Inhibition of the adenosinergic pathway using the A2Ar antagonist istradefylline resulted in abrogation of suppression and increase in antigen specific T cell proliferation (FIG. 10). This suggests that the regulatory function of the T cell lines is mediated through upregulation of CD39 and CD73, which leads to generation of adenosine and activation of the adenosinergic pathway. Furthermore, the upregulation of surface PD-1 expression on the T cell lines, another cell surface marker associated with activation of the adenosinergic signaling pathway, was detected (FIG. 11).

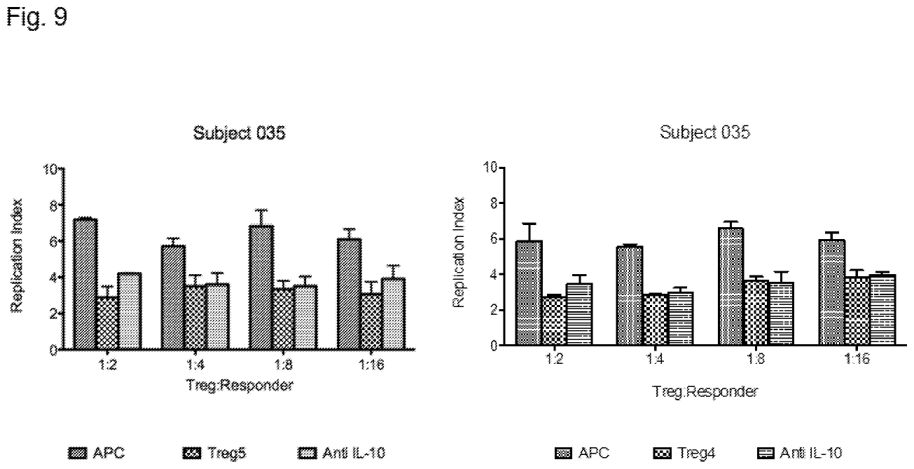

To determine if IL-10 contributes to the suppressive mechanism of the T cell lines, neutralizing IL-10 monoclonal antibody was used in a standard suppression assay. There was no change in the suppression of antigen specific T cell proliferation in the presence or absence of neutralizing IL-10 monoclonal antibody by the ex vivo expanded T cell lines (FIG. 9). Furthermore, TGF-β neutralizing antibody also did not have any effect on the immunosuppressive ability of the T cell lines. It was previously reported previously that IL-10 and TGF-β neutralizing antibodies together at a high concentration can abrogate Treg mediated suppression. However, no change in the T cell line mediated suppression was observed in the presence of both IL-10 and TGF-β neutralizing antibodies in a standard suppression assay.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. An isolated regulatory T cell (Treg) comprising a T cell receptor (TCR) that specifically binds to:
(i) an alloantigen that is a human leukocyte antigen (HLA) molecule, or a fragment thereof, and is not encoded by a nucleotide sequence present in the genome of the Treg, or
(ii) an autoantigen contributing to an autoimmune disorder, or a fragment thereof.

2. The Treg of claim 1, wherein the TCR specifically binds to the HLA molecule.

3. The Treg of claim 2, wherein the TCR specifically binds to a hypervariable region (HVR) of the HLA molecule.

4. The Treg of claim 3, wherein the TCR specifically binds to a β-chain HVR of the HLA molecule.

5. The Treg of claim 2, wherein the HLA molecule is an HLA-DR, HLA-DQ, HLA-DP, HLA-A, HLA-B, or HLA-C molecule, or a fragment thereof.

6. The Treg of claim 5, wherein the HLA molecule is an HLA-DR, HLA-DQ, or HLA-DP molecule, or a fragment thereof.

7. The Treg of claim 6, wherein the HLA-DR molecule is an HLA-DR1, HLA-DR2, HLA-DR3, HLA-DR4, HLA-DR5, HLA-DR6, HLA-DR7, HLA-DR8, HLA-DR9, HLA-DR10, HLA-DR11, HLA-DR12, HLA-DR13, HLA-DR14, HLA-DR15, HLA-DR16, HLA-DR17, HLA-DR18, HLA-DR51, HLA-DR52, or HLA-DR53 molecule, or a fragment thereof.

8. The Treg of claim 1, wherein the Treg is capable of suppressing T effector cell (Teff) responses directed towards the alloantigen or the autoantigen.

9. The Treg of claim 8, wherein the Treg is capable of suppressing Teff proliferation responses to direct allorecognition, semi-direct allorecognition, and/or indirect allorecognition.

10. The Treg of claim 8, wherein the Treg is capable of activating the adenosinergic signaling pathway.

11. The Treg of claim 1, wherein the Treg expresses one or more markers selected from the group consisting of CD4, CD25, CD39, CD73, FOXP3, GITR, CLTA4, ICOS, GARP, LAP, PD-1, CCR6, and CXCR3.

12. The Treg of claim 2, wherein the HLA molecule, or the fragment thereof, to which the TCR specifically binds is encoded by a nucleotide sequence that is present in the genome of a donor of an organ or tissue.

13. An isolated Treg comprising a TCR that specifically binds to:
(i) an alloantigen that is an HLA molecule, or a fragment thereof, and is not encoded by a nucleotide sequence present in the genome of the Treg, or
(ii) an autoantigen contributing to an autoimmune disorder, or a fragment thereof;
wherein the Treg has been produced by a method comprising:
(a) contacting an immune cell population comprising T cells obtained from a recipient subject with a fragment of the HLA molecule or autoantigen and an autologous antigen-presenting cell (APC); and
(b) expanding the immune cell population of step (a) for a time and under conditions sufficient to form an expanded T cell line comprising a plurality of the Tregs; and, optionally
(c) purifying the Tregs from the immune cell population.

14. The Treg of claim 13, wherein the immune cell population of (a) further comprises natural killer (NK) cells, and, if step (c) is performed, step (c) comprises purifying the Tregs and NK cells from the immune cell population, thereby producing a mixed population of Tregs and NK cells.

15. A mixed population of cells comprising the Treg of claim 1 and NK cells.

16. A composition comprising the Treg of claim 1.

17. A composition comprising a mixed population of cells comprising the Treg of claim 1 and NK cells.

18. A method of suppressing an immune response in a subject, the method comprising administering the pharmaceutical composition of claim 16 or 17 to the subject.

19. The method of claim 18, wherein the immune response is a Teff response directed towards the alloantigen or the autoantigen.

20. A method of treating or preventing transplant rejection or a method of treating an autoimmune disorder in a subject, the method comprising administering the composition of claim 16 or 17 to the subject.

21. The method of claim 18, wherein the subject has an autoimmune disorder.

22. The method of claim 18, wherein the subject is an organ or tissue transplant recipient.

23. The method of claim 18, wherein the HLA molecule, or the fragment thereof, to which the TCR specifically binds is encoded by a nucleotide sequence that is present in the genome of the donor of the organ or tissue.

24. The method of claim 18, wherein the method further comprises reducing the dose of an immunosuppressive agent administered to the subject.

25. The method of claim 18, wherein the organ is a kidney, a liver, a heart, a lung, a pancreas, an intestine, a stomach, a testis, a penis, a thymus, or a face, hand, or leg vascular composite allograft.

26. The method of claim 18, wherein the tissue comprises bone, a tendon, a cornea, skin, a heart valve, nervous tissue, bone marrow, islets of Langerhans, stem cells, blood, or a blood vessel.

27. The method of claim 20, wherein the autoimmune disorder is autism, autism spectrum disorder, rheumatoid arthritis, lupus, focal segmental glomerulonephritis, or membranous nephropathy.

28. A method for producing the Treg of claim 1, the method comprising:
(a) contacting an immune cell population comprising T cells obtained from a recipient subject with a fragment of the HLA molecule or autoantigen and an autologous APC; and
(b) expanding the immune cell population of step (a) for a time and under conditions sufficient to form an expanded T cell line comprising a plurality of the Tregs; and, optionally
(c) purifying the Tregs from the immune cell population.

29. The method of claim 28, wherein the method comprises repeating steps (a) and (b) more than three times.

30. The method of claim 28, wherein the method comprises repeating steps (a) and (b) four or five times.

31. The method of claim 28, wherein step (a) is performed about every seven to ten days.

32. The method of claim 28, wherein the autologous APCs are peripheral blood mononuclear cells (PMBCs), dendritic cells, macrophages, or B cells.

33. The method of claim 32, wherein the autologous APCs are PBMCs.

34. The method of claim 33, wherein the PBMCs are irradiated.

35. The method of claim 28, wherein the immune cell population comprising T cells is a population of PMBCs, a population of naïve T cells, or a population of purified Tregs.

36. The method of claim 35, wherein the immune cell population is a population of PBMCs.

37. The method of claim 36, wherein step (a) further comprises contacting the population of PBMCs with IL-2.

38. The method of claim 37, wherein the concentration of IL-2 is about 50 IU/ml to about 200 IU/ml.

39. The method of claim 38, wherein the concentration of IL-2 is about 100 IU/ml.

40. The method of claim 28, wherein the concentration of the fragment of the HLA molecule or autoantigen is about 25 μg/ml to about 200 μg/ml.

41. The method of claim 40, wherein the concentration of the fragment of the HLA molecule or autoantigen is about 50 μg/ml.

42. The method of claim 28, wherein the fragment of the HLA molecule or autoantigen is a purified peptide or peptide mixture.

43. The method of claim 28, wherein the immune cell population comprises NK cells.

44. The method of claim 28, wherein step (c) comprises purifying the Tregs and NK cells from the immune cell population, thereby producing a mixed population of Tregs and NK cells.

45. A composition comprising:
(a) the Treg of claim 1; and
(b) a fragment of the HLA molecule or autoantigen.

46. The composition of claim 45, wherein the composition further comprises IL-2.

47. The composition of claim 46, wherein the concentration of IL-2 is about 50 IU/ml to about 200 IU/ml.

48. The composition of claim 47, wherein the concentration of IL-2 is about 100 IU/ml.

49. The composition of claim 45, wherein the concentration of the fragment of the HLA molecule or autoantigen is about 25 μg/ml to about 200 μg/ml.

50. The composition of claim 49, wherein the concentration of the fragment of the HLA molecule or autoantigen is about 50 μg/ml.

51. The composition of claim 45, wherein the fragment of the HLA molecule or autoantigen is a purified peptide or peptide mixture.

52. The composition of claim 45, further comprising NK cells.


SEQUENCE LISTING (15 sequences)

SEQ ID NO: 1 - protein, 21 aa, Homo sapiens
RFLWQLKFECHFFNGTTERVR

SEQ ID NO: 2 - protein, 21 aa, Homo sapiens
TERVRLLERCIYNQEESVRFD

SEQ ID NO: 3 - protein, 21 aa, Homo sapiens
SDVGEYRAVTELGRPDAEYWN

SEQ ID NO: 4 - protein, 18 aa, Homo sapiens
SQKDLLEQRRAAVDTYCR

SEQ ID NO: 5 - protein, 14 aa, Homo sapiens
HNYGVGESFTVQRR

SEQ ID NO: 6 - protein, 20 aa, Homo sapiens
GDTRPRFLWQPKRECHFFNG

SEQ ID NO: 7 - protein, 19 aa, Homo sapiens
ERVRFLDRYFYNQEESVRF

SEQ ID NO: 8 - protein, 20 aa, Homo sapiens
DSDVGEFRAVTELGRPDAEY

SEQ ID NO: 9 - protein, 20 aa, Homo sapiens
WNSQKDILEQARAAVDTYCR

SEQ ID NO: 10 - protein, 14 aa, Homo sapiens
HNYGVVESFTVQRR

SEQ ID NO: 11 - protein, 16 aa, Homo sapiens
RFLEYSTSECHFFNGT

SEQ ID NO: 12 - protein, 20 aa, Homo sapiens
ERVRYLDRYFHNQEENVRFD

SEQ ID NO: 13 - protein, 20 aa, Homo sapiens
DSDVGEFRAVTELGRPDAEY

SEQ ID NO: 14 - protein, 18 aa, Homo sapiens
SQKDLLEQKRGRVDNYCR

SEQ ID NO: 15 - protein, 14 aa, Homo sapiens
HNYGVVESFTVQRR